(19) United States
(12) Patent Application Publication
(10) Pub. No.: US 2024/0408367 A1
(43) Pub. Date: Dec. 12, 2024

(54) MICRONEEDLE, MICRONEEDLE PATCH, METHOD OF MANUFACTURE AND METHOD OF USE THEREOF

(71) Applicant: City University of Hong Kong, Kowloon (HK)

(72) Inventors: Chenjie Xu, Kowloon Tong (HK); Tianli Hu, Xi'an (CN)

(21) Appl. No.: 18/735,676

(22) Filed: Jun. 6, 2024

Related U.S. Application Data
(60) Provisional application No. 63/576,460, filed on Jun. 7, 2023.

(51) Int. Cl. A61M 37/00; A61B 10/00; A61F 9/00; B29C 35/16; B29C 39/00; B29C 39/02; B29C 39/38; B29K 105/00; B29K 105/20; B29K 705/00; B29L 31/00

(52) U.S. Cl. CPC A61M 37/0015; A61B 10/0045; A61F 9/0017; B29C 35/16; B29C 39/003; B29C 39/006; B29C 39/026; B29C 39/38; A61B 2010/0067; A61M 2037/003; A61M 2037/0053; A61M 2210/0625; B29C 2035/165; B29K 2105/0002; B29K 2105/20; B29K 2705/00; B29K 2905/00; B29L 2031/7544

(57) ABSTRACT

A microneedle includes a base and a tip distal from the base, the microneedle being formed with an anisotropic porous composition including a plurality of channels extending in a substantially uniform direction through the microneedle from a base surface towards an outer surface defined by the tip, and the plurality of channels being adapted to enable flow of a fluid therein.

### FIELD OF THE INVENTION

The present invention relates to a microneedle, a microneedle patch, method of manufacture and method of use thereof. More particularly, the present invention relates to a microneedle and a microneedle patch comprising a plurality of uniform channels for facilitating fluid flow therein, a method of manufacture and a method of using the same.

### BACKGROUND OF THE INVENTION

Microneedles (MNs) are minimized needle arrays and are well known in the art for a variety of uses such as, for example, drug delivery, cell delivery, etc. to/through the dermis and other locations. A few MN platforms have been developed for delivering cells.

While porous microneedles exist in the prior art, such pores within the microneedles are typically random in size and direction, and are sponge-like with pores leading to many dead ends and closed cell structures, thus limiting the efficiency of drug or cell delivery. US 2016/158511 A1 (US '511), entitled “Fabrication of Phase-Transition microneedle patch”, to Jin, published on 9 Jun. 2016, describes a porous MN and its fabrication. However, the MN in US '511 contains randomly oriented interconnected pores that are not channels. Furthermore, it is believed that the diameter of these interconnected pores is not uniform, but is distributed within a range.

Further, previous methods to produce and/or load microneedles with their payload(s) often employs centrifugation which is a violent and high-gravity method. As certain cells and molecules to be delivered by a MN may be quite delicate, such centrifugation may damage and/or corrupt certain cells if they are the intended payload.

There thus remains a need for microneedles which provide easier manufacture, easier payload loading, reduced payload destruction/inactivation, unidirectional flow to reduce potential cross-contamination, provide anisotropic properties, and/or accurately deliver the anisotropic porous MN's payload. Furthermore, it has been found that it would be desirable to provide a manufacturing and/or loading method for preparing cells for transdermal cell delivery which is essentially free of payload centrifugation. There also remains a need for a method and article for sampling fluids, such as tears, which does not rely on, for example, Schirmer strips and/or hard materials such as capillary glass tubes.

### SUMMARY OF THE INVENTION

In a first aspect, there is provided a microneedle comprising a base and a tip distal from the base, wherein the microneedle is formed with an anisotropic porous composition comprising a plurality of channels extending in a substantially uniform direction through the microneedle from a base surface towards an outer surface defined by the tip, and wherein the plurality of channels are adapted to enable flow of a fluid therein.

In some embodiments, each of the plurality of channels has a substantially uniform channel diameter.

In some embodiments, the channel diameter is about 20 μm; or about 30 μm; or between 20-50 μm; or between 20-150 μm; or between 85-145 μm.

In some embodiments, the flow of the fluid within the plurality of channels is unidirectional.

In some embodiments, the plurality of channels has a first physical property when the fluid flows into the microneedle, and a second physical property when the fluid flows out of the microneedle, and wherein the first physical property is different from the second physical property.

In some embodiments, the first and second physical properties include morphological property, mechanical property, pore structure, and young's modulus.

In some embodiments, the anisotropic porous composition is formed with a cross-linked polymeric matrix.

In some embodiments, the cross-linked polymeric matrix includes a plurality of monomers selected from a group consisting of gelatin, alginate, polyvinyl alcohol (PVA), poly 2-hydroxyethylmethacrylate (PHEMA), polyacrylamide (PAAm), vinylgroup-modified hyaluronic acid (HA), methactylated hyaluronic acid (MeHA) and a combination thereof.

In a second aspect, there is provided a microneedle patch comprising a substrate and at least a microneedle disposed on the substrate.

In some embodiments, the substrate is unitary formed with the microneedle, and wherein the plurality of channels extend through both the substrate and the microneedle, from an outer surface of the substrate towards the outer surface defined by the tip of the microneedle.

In some embodiments, the substrate is separately formed with the microneedle, and is attached to the base of the microneedle.

In some embodiments, the substrate is a metal sheet.

In some embodiments, the substrate is a handle.

In some embodiments, the microneedle patch further comprising an electrochemical sensor attached to the substrate, and, in some embodiments, a payload pre-loaded within the microneedle patch.

In some embodiments, the payload is selected from a group consisting of a cell, a drug, a macromolecule, an extracellular vesicle, and a combination thereof.

In some embodiments, the cell is selected from a group consisting of an immune cell, an antigen cell, a stem cell, a melanocyte, a hair follicle cell, a beta cell, a fibroblast, a therapeutic cell, a prophylactic cell, and a combination thereof.

Extracellular vesicle (EVs) are cell-derived membrane-surrounded vesicles that carry bioactive molecules and deliver them to recipient cells. In some embodiments, the extracellular vesicle is selected from a group consisting of an exosome, a micro vesicle, an apoptotic body, an autophagic extracellular vesicle, a matrix vesicle, a stressed extracellular vesicle, and a combination thereof.

In some embodiments, the macromolecule is selected from a group consisting a genetic material, a polypeptide, a protein, a deoxyribonucleic acid sequence (DNA), a ribonucleic acid sequence (RNA), an enzyme, an antibody, and a combination thereof.

In some embodiments, the microneedle patch further comprising a plurality of microneedles forming a microneedle array disposed on the substrate.

In a third aspect, there is provided a method of manufacturing a microneedle, comprising the steps of: (i) casting a pre-polymer solution comprising a plurality of monomers into a mold defined by at least a recess shaped in a microneedle structure, wherein the microneedle structure comprising a base and a tip distal from the base; (ii) freezing the pre-polymer solution with a temperature gradient across the mold; (iii) cross-linking the plurality of monomers to form a cross-linked polymer matrix; (iv) lyophilising the cross-linked polymer matrix to form an anisotropic porous composition defined with the microneedle structure, wherein the anisotropic porous composition comprising a plurality of channels extending in a substantially uniform direction through the anisotropic porous composition from a base surface towards the outer surface of defined by the tip; and (v) removing the anisotropic porous composition from the mold.

In some embodiments, the temperature gradient is provided by arranging a first surface of the mold at room temperature; or at a temperature between 4 to 28° C.; or at a temperature between 18 to 24° C., and further arranging a second surface of the mold opposite the first surface at a temperature between −0 to −300° C.; or at a temperature between −25 to −285° C.; or at a temperature between −50° C. to −250° C.

In some embodiments, the second surface of the mold is placed in contact with a cooling element selected from a group consisting of liquid nitrogen, liquid helium, dry ice, and a mixture thereof.

In some embodiments, the cooling element is in form of a cooling bath.

In some embodiments, the cross-linking of the plurality of monomers is achieved by freezing the pre-polymer solution at a temperature below zero.

In some embodiments, the cross-linking of the plurality of monomers is achieved by exposing the pre-polymer solution to a UV source.

In some embodiments, the method further comprising a step of thawing the cross-linked polymer matrix at room temperature before the lyophilizing step (iv).

In some embodiments, the cross-linked polymer matrix is thawed in deionized water for an 1 hour to 3 days; or for 4 hours to 2 days; or for 6 hours to 36 hours.

In some embodiments, the mold is formed with a mold material selected from a first group consisting of a polymer, a metal, and a combination thereof; or from a second group consisting of polydimethylsiloxane (PDMS), steel, and a combination thereof.

In some embodiments, each of the plurality of channels has a substantially uniform channel diameter.

In some embodiments, the method further comprising a step of adjusting the channel diameter by varying a polymer concentration of the pre-polymer solution.

In some embodiments, the method further comprising a step of adjusting the channel diameter by varying the temperature gradient.

In some embodiments, the method further comprising a step of adjusting the channel diameter to about 20 μm; or about 30 μm; or between 20-50 μm; or between 20-150 μm; or between 85-145 μm.

In some embodiments, the plurality of monomers are selected from a group consisting of gelatin, alginate, polyvinyl alcohol (PVA), poly 2-hydroxyethylmethacrylate (PHEMA), polyacrylamide (PAAm), vinylgroup-modified hyaluronic acid (HA), methactylated hyaluronic acid (MeHA) and a combination thereof.

In a fourth aspect, there is provided a method of manufacturing a microneedle patch, comprising the steps of the manufacturing method of a microneedle, and further comprising a step of forming a substrate on which at least a microneedle is disposed.

In some embodiments, the substrate is formed by casting the pre-polymer solution into the mold beyond filling the recess of the mold along a mold wall.

In some embodiments, the substrate is formed by attaching a separate layer to the base of the microneedle.

In some embodiments, the separate layer is a metal sheet.

In some embodiments, the separate layer is a handle.

In some embodiments, the method further comprising a step of loading a payload to the microneedle patch.

In some embodiments, the payload is selected from a group consisting of a cell, a drug, an extracellular vesicle, a macromolecule, and a combination thereof.

In some embodiments, the cell is selected from a group consisting of an immune cell, an antigen cell, a stem cell, a melanocyte, a hair follicle cell, a beta cell, a therapeutic cell, a fibroblast, a prophylactic cell, and a combination thereof.

In some embodiments, the extracellular vesicle is selected from a group consisting of an exosome, a micro vesicle, an apoptotic body, an autophagic extracellular vesicle, a matrix vesicle, a stressed extracellular vesicle, and a combination thereof.

In some embodiments, the macromolecule is selected from a group consisting a genetic material, a polypeptide, a protein, a deoxyribonucleic acid sequence (DNA), a ribonucleic acid sequence (RNA), an enzyme, an antibody, and a combination thereof.

In some embodiments, the payload is loaded to the pre-polymer solution when the polymer solution is casted in the mold.

In some embodiments, the payload is loaded to the anisotropic porous composition, followed by a further step of attaching the substrate to the base of the microneedle.

In some embodiments, the method further comprising a step of attaching an electrochemical sensor to the substrate.

In a fifth aspect, there is provided a method of use of a microneedle patch, comprising a step of applying the microneedle patch to an area of application to deliver the payload from the microneedle patch to the area of application.

In some embodiments, the area of application is a skin surface.

In some embodiments, the area of application is an ocular surface

In some embodiments, the area of application is an oral surface.

In a sixth aspect, there is provided a method of use of a microneedle patch, comprising a step of contacting a sample with the microneedle patch to collect the sample.

In some embodiments, the sample is a tear.

### BRIEF DESCRIPTION OF THE DRAWINGS

In order that a more precise understanding of the above-recited invention can be obtained, a more particular description of the invention briefly described above will be rendered by reference to specific embodiments thereof that are illustrated in the appended drawings. The drawings presented herein may not be drawn in scale and any reference to dimensions in the drawings or the following description is specific to the embodiments disclosed.

FIG. 1 shows a schematic view of an embodiment of a microneedle (MN) patch according to the present invention;

FIG. 2 shows a partially cut-away side view of a MN patch according to an embodiment of the present invention;

FIG. 3 shows a close-up schematic diagram of an embodiment of a microneedle according to the present invention;

FIG. 4 shows a schematic view of a mold according to an embodiment of the present invention when the mold is filled with a polymer solution;

FIG. 5 shows a schematic view of a filled mold according to FIG. 4;

FIG. 6 shows a schematic view of a filled mold similar to that in FIG. 5, being frozen according to an embodiment of the present invention;

FIG. 7 shows a schematic view of an embodiment of the MN array removed from the mold;

FIG. 8 shows a schematic view of an embodiment of the manufacturing method according to the present invention;

FIG. 9 shows a schematic view of an embodiment of the manufacturing method according to the present invention where the payload is added to the pre-polymer solution while the pre-polymer solution is in the mold;

FIG. 10 shows a schematic view of an embodiment of a method of use of a MN patch according to the present invention;

FIG. 11 shows a schematic view of a monitoring system;

FIG. 12 shows a schematic diagram of an embodiment of a microneedle according to the present invention for use in transdermal cell delivery and tear extraction;

FIG. 13, at part a, shows a schematic view of an embodiment of a manufacturing method according to the present invention; at part b, shows representative scanning electron microscopic (SEM) images of an anisotropic porous crystal gel (APC-), an isotropic porous crystal gel (IPC-), a lyophilized hydrogel (LH-) and a dried hydrogel (SH-) microneedle from a cross section view; at part c, shows a longitudinal section and cross section view of a microneedle according to an embodiment of the present invention; and, at part d, shows representative fluorescence images from a longitudinal view of a frozen sectioned APC-, IPC-, and LH-MN as shown in part b;

FIG. 14a shows SEM images of APC-, IPC-, LH-, and SH-cylindrical-shaped materials from a cross section view and a longitudinal section view;

FIG. 14b shows SEM images of APC-MNs with polymer concentrations of 2.5 wt. %, 5 wt. %, 7.5 wt. % and 10 wt. % fabricated with freezing temperatures at −20° C., −80° C. and −196° C., from a longitudinal section view;

FIG. 14c shows a flow process of a Cy3 dye solution in APC-MNs from the tip to the substrate with polymer concentrations of 2.5 wt. %, 5 wt. %, and 7.5 wt. %; and in IPC-MNs, LH-MNs, and solid hydrogel (SH-MD) with polymer concentration of 7.5 wt. %;

FIG. 15, at part a, shows a schematic view of an embodiment of a tear extraction process and post analysis using an APC-MN according to the present invention; at part b, shows a graph indicating the results of a mechanical test of APC-MNs with a horizontal force (shear force) applied to a top of the MNs; at part c, shows ematoxylin and eosin (H&E) stain images of an untreated eye of a mouse and its eyes treated with APC-MNs and SH-MNs; at part d, shows a graph indicating the recovery percentage of FITC-Dextran (10 kDa) from MNs, commercial strips, and cotton swabs respectively; and at parts c and d, shows graphs indicating (e) the recovery ratio and (f) the IFN-gamma from APC-MNs with a polymer concentration of 5 wt. %, commercial strips, and swabs, respectively;

FIG. 16, at part a, shows a schematic view of an embodiment of a manufacturing method of an APC-Cryomicroneedle (cryoMN) according to the present invention; at part b, shows optical images indicating water contact angles on a PDMS negative microneedle molds including unpacked (untreated), unpacked but hydrophilic treated mold (plasma-treated), and APC-MN packed (APC-MN filled) molds, respectively, for measuring the water contact angle, at part c, shows a graph demonstrating the quantified water contact angle on a PDMS negative microneedle molds including unpacked (untreated), unpacked but hydrophilic treated mold (plasma-treated), and APC-MN packed (APC-MN filled) molds; at part d, shows images of the Cy3 dye distribution in the tip holes of a PDMS negative microneedle molds including unpacked (untreated), unpacked but hydrophilic treated mold (plasma-treated), and APC-MN packed (APC-MN filled) molds; at part e, shows a graph indicating fluorescence distribution within PDMS negative microneedle molds including unpacked (untreated), unpacked but hydrophilic treated mold (plasma-treated), and APC-MN packed (APC-MN filled) molds; at part f, shows optical images demonstrating morphology of an APC-Cryomicroneedles (APC-cryoMN); at part g, shows a close-up image of an aligned porous structure of an APC-CryoMN after melting in PBS; and, at part h, shows a statistical graph demonstrating water contact angles of PDMS negative microneedle molds including unpacked (untreated), unpacked but hydrophilic treated mold (plasma-treated), and APC-MN packed (APC-MN filled) molds;

FIG. 17a shows distribution of fluorescence dye with different molecular weight (Cy3 and FITC-dextrans with 10 kDa and 150 kDa molecular weights or FD1W and FD15W) in the tip of APC2.5-, APC5-, IPC5-, LH5-, and APC7.5-MNs that stayed in the PDMS mold;

FIG. 17b shows FITC-LNP distribution in the tip of APC2.5-, APC5-, IPC5-, and APC7.5-MNs that stayed in the PDMS mold;

FIG. 18 shows photographic images of demolded APC-cryoMNs whose base is made of various base formulations, including PBS, PBS containing 10 wt. % 10 kDa HA, and PBS with 10 wt. % 13 kDa PVA;

FIG. 19 shows penetration of hMSC, DC, 3T3, B16, and C2C12 cells in APC-MNs that stayed in the PDMS molds;

FIG. 20, at parts a and b, shows optical images and thermal images of a microneedle patch according to an embodiment of the present invention where the microneedle patch is in a thawing process at room temperature; at part c, shows a graph demonstrating temperature change profile over time of a microneedle patch of part b; at part d, shows thermal images and optical images of a microneedle patch of parts a and b, where the microneedle patch is in a thawing process at skin temperature; at part e, shows a graph demonstrating a temperature change profile over time of a microneedle patch of part d; at part f, shows a graph demonstrating compression test results of cyroMNs (pure cryogenic medium), APC2.5-cryoMN, APC5-cryoMN, and APC7.5-cryoMN at a displacement speed of 0.5 mm/s; and, at part g, shows histological H&E images of porcine skin treated with APC5-cryoMN and untreated skin;

FIG. 21, at part a, shows γδ T cell distribution in the tips of an APC2.5-, APC5-, IPC5-, and APC7.5-MN when cells are loaded onto an APC-MN that stayed in the PDMS mold; at part b, shows a tip of a detached microneedle loaded with cells; at part c and d, shows graphs indicating cell viability of γδ T cells cryopreserved in (c) vial tubes and (d) APC-MNs; and, at part e, shows a graph indicating the percentage of Vδ2+ cells after recovering from APC-cryoMNs;

FIG. 22, at part a, shows optical images of posterior skin of a mouse applied with APC-cryoMN; at part b, shows representative flow cytometric dot plots of CD3⁺Vδ2⁺ cells in the spleen, lung, liver, and tumour of a mouse at 3 days post γδ T cells injection; at part c, shows graphs demonstrating the flow cytometry analysis of the frequency of CD3⁺Vδ2⁺ cells in the spleen, lung, liver and tumour of a mouse; and, at part d, shows representative immunostaining images of Vδ2⁺ cells in skin tissue of a mouse;

FIG. 23, at part a, shows luciferase activity of MSTO-luc mice in each treatment group during a MSTO-luc mice experiment; and, at part b, shows weight changes of MSTO-luc mouse in each treatment group during the experiment of part a;

FIG. 24, at part a, shows a timeline of an experiment involving MSTO-luc bearing NSG mice; at part b, shows representative images of luciferase activities of MSTO-luc bearing mice receiving Vδ2 T cell at 1-dose or 2-dose regimen from APC-cryoMN or intravenous injection (i.v.) routes; at part c, shows a graph indicating tumor growth measured by luciferase activities over time compared to day 2 post MSTO-luc tumor injection for different treatment groups; and, at part d, shows a Kaplan-Meier plot for MSTO-luc mice for different treatment groups over time;

FIG. 25, at part a, shows a timeline of an experiment involving A375-bearing NSG mice; at part b, shows photographic representations of the tumor volume of mice receiving PBS as control, Vδ2 T from i.v., and APC-cryoMN routes over a period of 11 days; at part c, shows a graph indicating the tumor volume extracted from a mouse from each treatment group over time; at part d, shows a graph demonstrating the weight change of mice in each treatment group over time; and, at part e, shows representative immunostaining images of Vδ2⁺ cells in melanoma tumor tissue and skin tissue.

### DETAILED DESCRIPTION OF PREFERRED EMBODIMENTS

### 1. Definitions

Unless otherwise specifically provided, all tests herein are conducted at standard conditions which include a room and testing temperature of 25° C., sea level (1 atm.) pressure, pH 7, and all measurements are made in metric units. Furthermore, all percentages, ratios, etc. herein are by weight, unless specifically indicated otherwise. It is understood that unless otherwise specifically noted, the materials compounds, chemicals, etc. described herein are typically commodity items and/or industry-standard items available from a variety of suppliers worldwide.

As used herein, the term “anisotropic” indicates that the physical properties of the porous microneedle change depending on the direction that materials are traveling through the channels in the microneedle. Examples of anisotropic properties herein include, for example, morphological properties, mechanical properties, pore structure, and/or young's modulus.

As used herein, the term “loaded” indicates that a payload is contained within the channel of the anisotropic porous MN for delivery to a target. Conversely, the term “unloaded” as used herein indicates that no payload is contained within the channel of the anisotropic porous MN. Such terms typically refer to a specific anisotropic porous MN, unless otherwise specifically indicated.

As used herein, the term “lyophilization” indicates a process including the steps of 1) freezing an item at atmospheric pressure, the item containing liquid, typically water, in a chamber, and 2) lowering the chamber pressure so as to cause the liquid to sublime from the frozen matrix, leaving the matrix structure intact. As used herein the term “lyophilized” describes an item which has undergone a lyophilization process. Furthermore, as used herein, the term lyophilization includes the phrase “freeze drying”.

As used herein the term “microneedle” or “MN” refers to a micro-sized needle. Such needles may have a height of from about 25 μm to about 2000 μm. They are generally in the form of an array or a patch, and may be made of various materials.

As used herein, the term “needle length” refers to the length from the base of the microneedle to the tip of the needles thereof.

As used herein, “unidirectional flow” indicates flow that only goes in one direction, either when delivered via the microneedle tip, or when a sample is extracted via the microneedle tip.

### 2. Microneedle Patch of the Present Invention

In an embodiment of the invention herein, an anisotropic porous microneedle (MN) contains a channel; or a plurality of channels, therein. The channel or plurality of channels provide(s) an anisotropic property. In an embodiment herein, the anisotropic porous MN is an absorbent anisotropic porous MN; or a tear collecting absorbent anisotropic porous MN; or a cell loading anisotropic porous MN. Without intending to be limited by theory, it is believed that such an anisotropic porous microneedle may be employed in, for example, drug delivery, liquid sample extraction, cell delivery, etc.

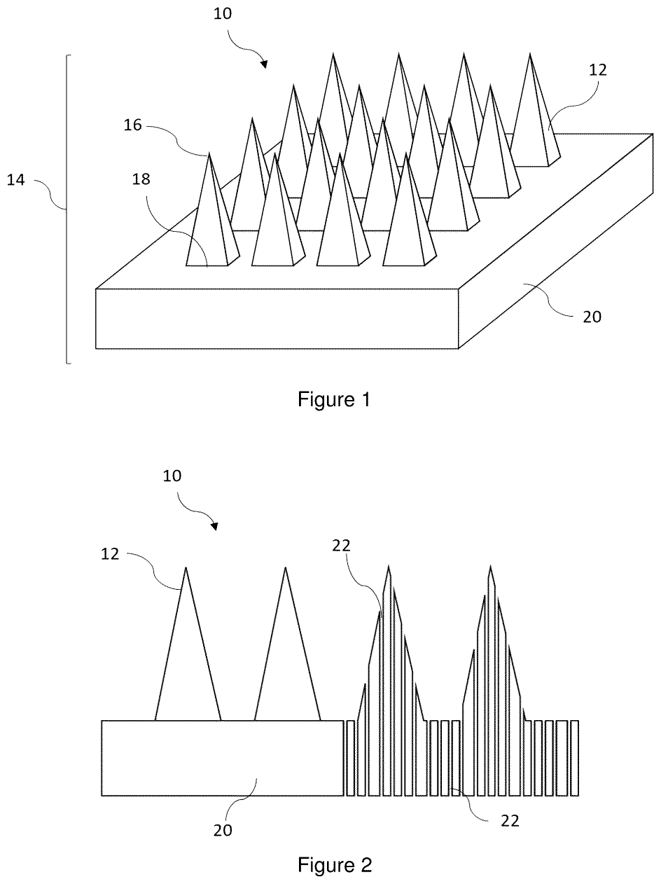

FIG. 1 is a schematic view of an embodiment of a microneedle patch 10 according to the present invention. The MN patch 10 contains a plurality of anisotropic porous MNs 12, formed by a polymeric matrix 14. Each of the anisotropic porous MNs 12 comprises a microneedle tip 16 at a distal from a microneedle base 18. The microneedle base 18 is integral with a substrate layer 20.

Without intending to be limited by theory, it is believed that while previous MNs possess pores, such pores are typically random in size, direction, and are like sponges with pores leading to many dead-ends and closed-cell structures. In contrast, it is believed that the present channels are significantly different from pores in that they have a diameter, a length and direction; or a significantly uniform diameter, a significantly uniform length, a significantly uniform direction, and a combination thereof. Furthermore, it is believed that the channels herein may be anisotropic in that their physical properties (e.g. morphology and mechanical properties) are different when liquid therein flows in different directions. The anisotropic channels in MNs may be formed by the manufacturing method described herein, which relies upon ice crystal growth during unidirectional freezing. In addition, it is believed that the material flow in the channels herein may be unidirectional.

In an embodiment herein, the channel has a channel diameter, and this channel diameter is substantially uniform, meaning that the channel diameter varies less than about ±25%; or from about 0% to about 25%; or from about 3% to about ±15%; or from about 5% to about 15%, along the length of the channel. Without intending to be limited by theory, it is believed that a uniform channel diameter provides benefits such as, for example, greater predictability, more consistent loading, consistent and predictable extraction and/or loading speed, batch-to-batch consistency, etc.

FIG. 2 shows a partially cut-away side view of a MN patch 10, according to an embodiment of the present invention. In FIG. 2, the MN patch is cross-sectionally cut in a longitudinal direction, showing that the MNs 12 contain a plurality of channels 22 therein. These channels 22 run from the substrate layer 20 to the MN base 18, and to the MN tip 16. The channels extend uniformly from the base of the substrate 20 to the tip of the microneedle 16 in a direction perpendicular to the substrate layer 20, due to the anisotropic structure of the microneedle patch 10.

Without intending to be limited by theory, it is believed that the MNs herein provide a variety of potential benefits over previous MNs such as, for example, unidirectional flow either into the MN or out of the MN, easier payload (i.e., a material or composition to be delivered) loading into the MN, easier sample (e.g., biological liquid) collection, reduced irritation and tissue damage during sample collection, channel size control/channel size tunability, a single manufacturing method to produce MNs useful in multiple different scenarios, uniform channel size, and a combination thereof.

In an embodiment herein, the anisotropic porous microneedle comprises; or is formed by, a cross-linked polymeric matrix. The cross-linked polymeric matrix forms the channel; or defines the channel edges, and provides unidirectional flow via, for example, capillary action. In an embodiment herein, the cross-linked polymeric matrix contains a monomer selected from the group consisting of, for example, gelatine, polyvinyl alcohol (PVA), poly 2-hydroxyethylmethacrylate (PHEMA), polyacrylamide (PAAm), vinylgroup-modified hyaluronic acid (HA) and a combination thereof; or gelatin, alginate, and a combination thereof. Typically, the cross-linked polymeric matrix is formed from a pre-polymer solution as described herein.

The cross-linked polymeric matrix herein contains a channel; or a plurality of channels; or channels having a substantially uniform diameter; or channels having a uniform diameter. The channel; or the channel edge, is defined by the edges of the polymeric matrix. Without intending to be limited by theory, it is believed that channels having a uniform diameter provide significant benefits such as, for example, uniform payload delivery, uniform sample collection, a soft and/or non-irritating feel to, for example, the eye surface, the location that the sample is extracted from, etc. Furthermore, without intending to be limited by theory, it is believed that the anisotropic porous MNs herein may be able to absorb a larger volume of liquid than a comparable previous MN. Furthermore, we believe that the anisotropic porous MNs' volume may not increase; or may remain substantially constant, even after absorbing the sample, and a combination thereof.

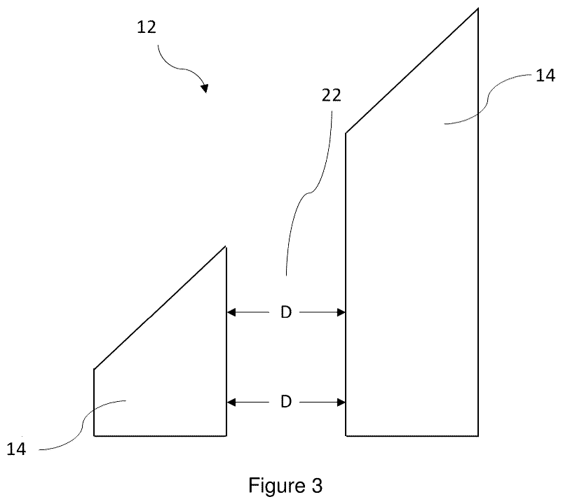

FIG. 3 shows a close-up schematic diagram of an embodiment of an anisotropic porous MN 12. The anisotropic porous MN 12 is formed by the polymeric matrix 14, which defines a channel 22 therein. The channel has a channel diameter, D, which is substantially uniform along the length of the channel 22 in the anisotropic porous MN 12.

Typically, the channel diameter useful herein ranges from larger than about 20 μm; larger than about 30 μm; or about 20 μm to about 150 μm; or from about 85 μm to about 145 μm. In an embodiment herein, the channel diameter is from about 20 μm to about 50 μm, especially when the anisotropic porous MN is to be used for drug delivery (i.e., the payload is a drug, especially a small molecule drug). Alternatively, if the payload to be delivered is a cell, then in an embodiment herein the channel diameter is from about 85 μm to about 145 μm, depending on the size of the cell need to be delivered.

The cell useful herein as a payload may be, for example, an immune cell, an antigen cell, a stem cell, a fibroblast, a melanocyte, a hair follicle cell, a beta cell, and a combination thereof. In an embodiment herein the cell is a therapeutic cell, a prophylactic cell, and a combination thereof. The cell herein may be a human cell, an animal cell, a plant-cell, a chimeric cell; or a human cell, human-derived cell, and a combination thereof. As human cells are typically from about 5 μm to about 30 μm in diameter, when the payload contains such a human cell, then the channel size should be at least about 30 μm to form a clear pathway for the cell to move from within the channel to the outside of the anisotropic porous MN.

Alternatively, the payload herein may include a cell, a drug, a macromolecule, and a combination thereof; or a genetic material, a bacterium, a virus, an animal cell, a cell extract, and a combination thereof; or a protein, DNA, RNA, an EVs, a human cell, an animal, non-human cell, a chimeric cell, a vector, and a combination thereof; or a cell; or a drug. The macromolecule may be a genetic material, a polypeptide, and a combination thereof; or a protein, a deoxyribonucleic acid sequence (DNA), a ribonucleic acid sequence (RNA), or a combination thereof; or an enzyme, an antibody, and a combination thereof.

Furthermore, it is believed that by pre-loading the MNs with the payload, and then lyophilizing the (pre-loaded) MN, this may protect the MN and the payload from adverse effects, such as, structural collapse, cell inactivation, efficacy loss, denaturation, degradation, etc. during transport, storage, etc.

In an embodiment herein, a microneedle array contains a plurality of MNs; or from about 1 MNs to about 1000 MNs; or from about 10 MNs to about 500 MNs; or from about 100 MNs to about 250 MNs; or from about 1 MNs to about 100 MNs. In an embodiment herein, the density of the MNs in a microneedle array is determined by the size of the MNs. Typically, the density of the MNs is from about 1 MNs per centimetre to about 20 MNs per centimetre. Such MNs are typically formed together as an integrated MN array.

Without intending to be limited by theory, it is believed that the anisotropic porous MN herein, or the MN array herein, may be very flexible and applicable to many different uses. For example, the anisotropic porous MN herein may provide consistent payload delivery if it is pre-loaded with a payload, or when not pre-loaded with a payload, it may provide consistent sample absorption and/or efficient retrieval in microliter quantities.

### 3. Method of Manufacture

In an embodiment herein the anisotropic porous MN may be formed by a manufacturing method including the steps of providing a pre-polymer solution containing a plurality of monomers therein, providing a mold containing a mold top surface and a mold bottom opposite the mold top surface, casting the pre-polymer solution into the mold, providing a temperature gradient between the mold top surface and the mold bottom, freezing the pre-polymer solution with the temperature gradient, cross-linking the plurality of monomers to form a cross-linked polymer matrix in the mold, and lyophilizing the MN scaffold to form an anisotropic porous MN.

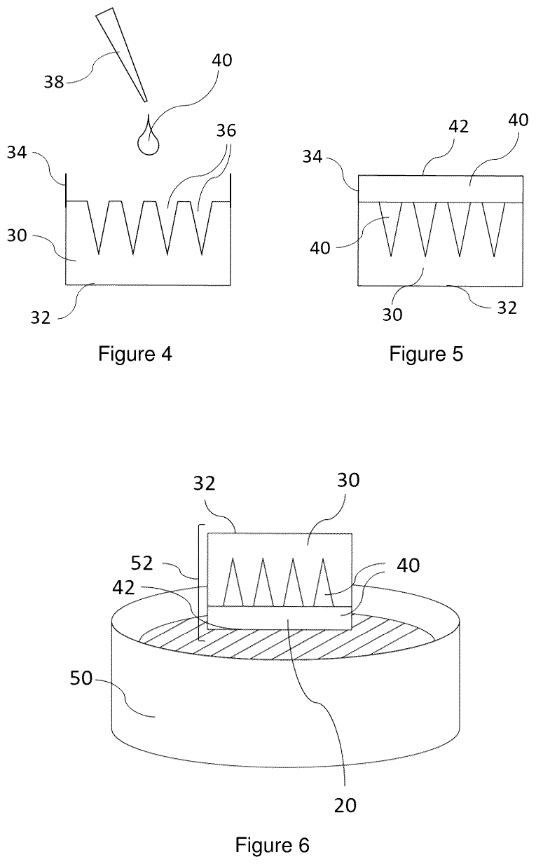

FIG. 4 shows a schematic view of a mold 30 according to an embodiment of the present invention. The mold 30 comprises a mold top surface 32, a mold wall 34 and a plurality of recesses 36, which are the negative impressions from which to form the MNs 12 (see FIG. 1). Furthermore, an applicator 38 is shown which applies the pre-polymer solution 40 into the mold 30, thereby casting the pre-polymer solution 40 into the mold. It can be seen that the mold wall 34 in this case defines the perimeter of the mold 30, allowing the mold 30 to be deeper than just the depth of the recesses 36. This allows the formation of the substrate layer (see FIG. 1 at 20), by filling the mold 30 past the point where the recesses 36 are filled, and ensuring that the substrate layer (see FIG. 1 at 20) is integral with the MNs (see FIG. 1 at 12).

Referring now to FIG. 5 which shows a schematic view of a filled mold 30 of FIG. 4. Once the mold 30 is filled with the pre-polymer solution 40, the mold bottom 42, is affixed to the mold, 30, opposite the mold top surface, 32. The outer surface of the substrate 42 forms a water-tight seal with the mold wall 34. In FIG. 5, it can be seen that the pre-polymer solution 40 fills the recesses 36 as well as the mold area adjacent to the mold walls 34. This ensures that sufficient pre-polymer solution 40, is present so as to integrally-form the substrate layer 20.

FIG. 6 shows a schematic view of an embodiment of a filled mold 30, similar to that in FIG. 4, being frozen according to an embodiment of the invention. The filled mold 30 is inverted with respect to FIG. 4, and the mold bottom 42 is placed on a cooling element 50. The substrate layer 20 is sealed by a mold bottom 42 which in this case is a sheet of tin foil, so as to prevent the pre-polymer solution 40 from leaking out of the mold 30. The mold top surface 32 is in contact with the air at room temperature. Accordingly, the mold top surface 32 and the mold bottom 42 are subjected to a temperature gradient 52.

The temperature gradient is formed by the difference in the of the mold top surface temperature and the mold bottom temperature. In an embodiment herein, the mold top surface is at room temperature; or from about 4° C. to about 28° C.; or from about 18° C. to about 24° C. In an embodiment herein the mold bottom temperature is from about −0° C. to about −300° C.; or from about −10° C. to about −285° C.; or from about −15° C. to about −250° C. For example, if the mold top surface is at room temperature of 25° C. and the mold bottom is on a cooling plate in liquid nitrogen at −196° C., then the temperature gradient is 221° C.

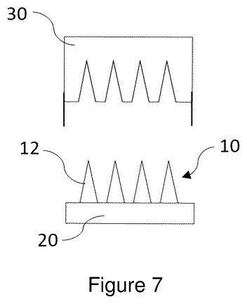

FIG. 7 shows a schematic view of the MN patch 10 being removed from the mold 30. After curing and either before or after loading, the MN patch 10 is removed from the mold 30 to release the MNs 12 attached to, and integral with, the substrate layer 20.

Typically, the mold herein is a negative mold, typically formed from a flexible, low-temperature-resistant material such as a polymer, a metal, and a combination thereof, or polydimethylsiloxane (PDMS), steel, copper, resin, and a combination thereof.

An embodiment of the method herein further contains the optional step of removing the anisotropic porous MN from the mold. In an embodiment of the invention, the anisotropic porous MN; or the MN array containing a plurality of anisotropic porous MNs, is simply peeled off of the mold.

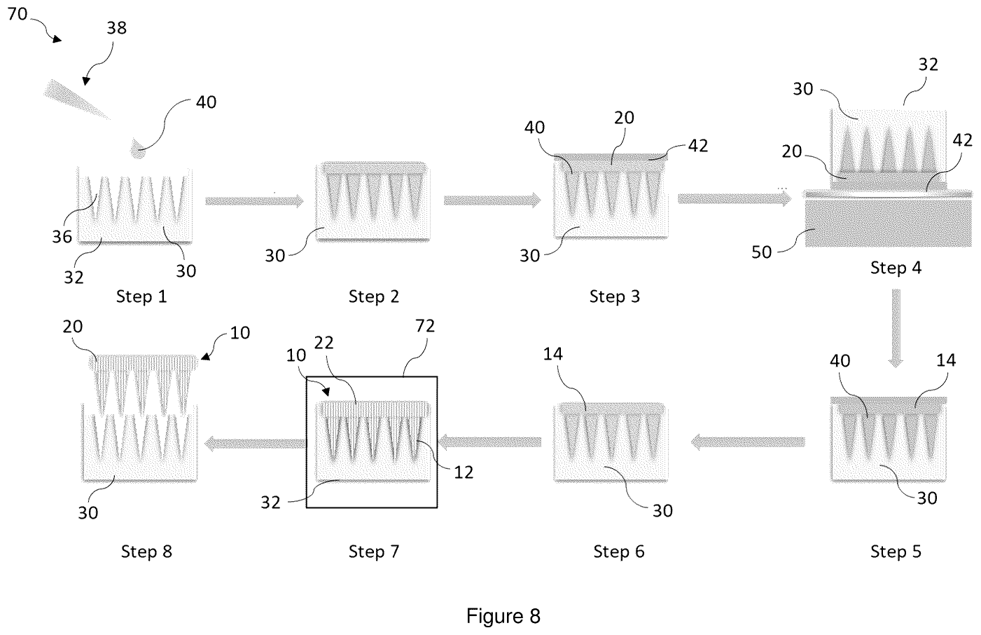

FIG. 8 shows a schematic view of an embodiment of the manufacturing method 70 according to the present invention. In Step 1 the pre-polymer solution 40 is provided in an applicator 38, which in this case is a plastic or glass dropper. A mold 30 is provided having a mold top surface 32 and an open mold bottom 42. The applicator 38 casts the pre-polymer solution 40 to the mold 30 by applying it thereto. The mold 30 is centrifuged at 4,000 rpm for 3 min with the recesses 36 pointing outwards to remove any air bubbles which may be present to prevent the MN tip (see FIG. 1 at 16) from forming. This results in the filled mold 30, shown in Step 2.

In Step 3, a substrate layer 20, in this case formed of tin foil, is added to the mold bottom 42 so as to seal the pre-polymer solution 40 into the mold 30. In Step 4, the mold 30 is inverted so that the mold bottom 42 is directly placed onto the cooling element 50, which contains liquid nitrogen at about −196° C. The mold top surface 32 is at room temperature of 25° C., thus providing a temperature gradient of 225° C. This results in unidirectional growth of channels from the mold bottom 42, towards the mold top surface 32.

In Step 5, the pre-polymer solution 40, still in the mold 30, is cross-linked in a sub-zero freezer for at least 1 day; or from about 1 day to about 1 week; or from about 1.5 days to about 4 days, so as to maintain the formed channels and polymerize the pre-polymer solution's monomers to form the polymeric matrix 14. Alternatively, the cross-linking may be initiated by, for example, exposing the mold containing the frozen pre-polymer solution to a UV source.

In Step 6, the mold 30, now containing the polymeric matrix 14, is thawed at room temperature in, for example, deionized water; or thawed in deionized water for from about 1 hour to about 3 days; or from about 4 hours to about 2 days; or for about 6 hours to about 36 hours. Without intending to be limited by theory, it is believed that this extended thawing process in deionized water also removes any unreacted compounds from the polymeric matrix.

In Step 7, the mold 30 is placed in a lyophilizer 72, with the mold top surface 32 facing down, and lyophilized to form a MN array 10 containing the anisotropic porous MNs 12, further containing a plurality of channels 22. In Step 8, the MN array 10 is demolded from the mold 30 by pulling on the substrate layer 20.

Typically, the cross-linked polymeric matrix is formed from the cross-linking of the ingredients in the pre-polymer solution as described herein. In an embodiment herein, the pre-polymer solution is in the form of an aqueous solution, a hydrogel, and a combination thereof; or a cryogel. The pre-polymer solution useful herein contains, for example, a monomer, a catalyst, a radical initiator, and a combination thereof. The monomer herein may be a polymeric monomer, a polymeric subunit, and a combination thereof. In an embodiment herein, the polymerization reaction is via cryogelation, radical polymerization initiated by, for example, light treatment such as UV light, and a combination thereof. In an embodiment herein, the cryogelation process includes the steps of phase separation via ice crystal formation of the channels, cross-linking, polymerization and thawing to form a cryogel network with channels therein.

In an embodiment herein the pre-polymer solution further contains a monomer. As described herein, the pre-polymer solution contains a monomer; or a polymeric monomer, a polymeric subunit, and a combination thereof, therein which is cross-linked to form the cross-linked polymer matrix. In an embodiment herein, the monomer is selected from gelatin, polyvinyl alcohol (PVA), vinyl group-modified hyaluronic acid (HA), poly(ethylene glycol) diacrylate (PEGDA), and a combination thereof; or gelatin, alginate, methacrylated hyaluronic acid (MeHA), and a combination thereof. The monomers useful herein are easily available from a variety of suppliers worldwide.

Without intending to be limited by theory, it is believed that when the pre-polymer solution is frozen without the temperature gradient described herein, then only pores; or interconnected pores, and not unidirectional channels, form as the ice crystals grow randomly in both their direction and size.

Furthermore, in prior MNs, the pre-polymer solution's monomers may be crosslinked prior to freezing. In such a process, it is believed that the porous structure of the polymers are again randomly-oriented and trap water (or whatever solvent is being used) and other pre-polymer solution constituents therein in pores having substantially random orientations, sizes, shapes, etc.

In contrast, and without intending to be limited by theory, it is believed that the uniform channels in the present invention are the result of the present invention's controlled freezing step in the presence of the temperature gradient, that occurs prior to the cross-linking step. It is believed that the freezing of the pre-polymer solution in the presence of the temperature gradient causes ice crystals to form ice crystal channels having a substantially uniform; or uniform, diameter. Specifically, at a molecular level the thermodynamics of ice crystal formation pushes the monomers to the side of the ice crystals. The monomers form ice crystals in a specific and parallel orientation, and with ice crystals having a substantially uniform diameter.

After the pre-polymer solution is frozen with the temperature gradient, then the monomer; or a polymeric monomer, a polymeric subunit, is cross-linked to form a cross-linked polymer matrix within the mold. Thus, when the mold and the frozen contents thereof are subject to lyophilization, the ice from the pre-polymer solution; or an aqueous solution, a hydrogel, and a combination thereof; or a cryogel, sublimes, leaving a (hollow) channel formed and bounded by the cross-linked polymer matrix. The resulting channel typically has a substantially uniform diameter.

Furthermore, it is believed that by adjusting the temperature of the bottom mold surface (i.e., which causes the temperature gradient), the width of the channels can be tuned for specific diameters.

In an embodiment herein, the anisotropic porous MNs in a MN patch are linked together by a substrate layer formed during the freezing step, which typically is formed by the same cross-linked polymeric matrix as the anisotropic porous MNs, a different cross-linked polymeric matrix than the anisotropic porous MNs; a handle; and a combination thereof while the MN array is still in the mold. In an embodiment herein, when the substrate layer is a thin flexible material, it may be a commonly-available thin flexible material such as, tin foil (a.k.a., aluminium foil), and a combination thereof. Without intending to be limited by theory, it is believed that tin foil may be useful herein as it is easily available, cheap, tough, flexible, and provides quick heat transfer, etc.

The handle useful herein may be a 3D printed flat base with an appropriately-sized and shaped pole for holding by the hand. The base attached to the MN array, while the pole may be held in the user's hand.

In an embodiment herein, the handle is formed from, for example, plastic, and may be manufactured using a 3D printer. In an embodiment herein, the handle is formed from VeroClear™ available from Stratasys, Eden Prairie, MN, USA, using Fused Deposition Manufacturing, a type of 3D printing. The supporting handle may be used in place of a thin flexible material, or may be used in addition to such a thin flexible material, as desired. In an embodiment herein, the supporting handle provides a heat sink which reduces the chance of the frozen MNs from melting prior to application or being used to deliver a payload.

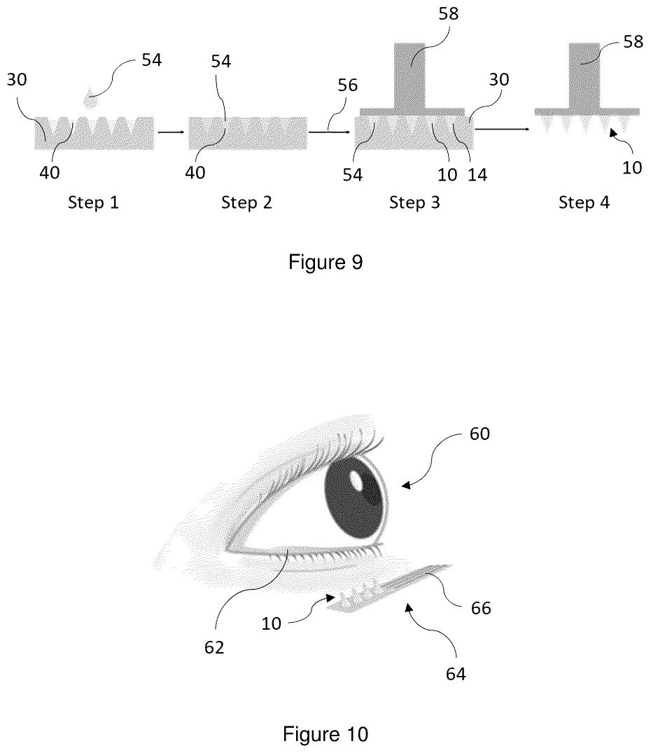

FIG. 9 shows a schematic view of a further embodiment of the manufacturing method herein. In Step 1, the payload 54 is added to the pre-polymer solution 40, while the pre-polymer solution 40 is in the mold 30. The mold 30 thus includes the mixed payload 54 and pre-polymer solution 40, and is subjected to a freezing step 56 and a cross-linking step (not shown) to form a polymeric matrix 14, already containing the payload 54. After the cross-linking step, a handle 58 is applied to the MN array 10 formed from the polymeric matrix 14. The handle 58 is used to pull the MN array 10, formed with the anisotropic porous structure, out of the mold 30, while still attached to the handle 58.

In an embodiment herein, the loading step occurs after freeze drying and while the anisotropic porous MN is still in the mold but without a substrate layer. Alternatively, in another embodiment herein, the loading step may occur after the pre-polymer solution is added to the mold and before the freezing step. In either embodiment, the payload may be provided as a payload solution and added, for example, dropwise, to the pre-polymer solution and/or the anisotropic porous MNs while they are still in the mold. Without intending to be limited by theory, it is believed that such methods may reduce potential waste of the payload in the backing layer, especially if the backing layer is formed of a polymer or connected monomers, such as those found in the pre-polymer solution. Such a benefit may be especially important when the payload is rare and/or expensive, such as in the case of drugs and autologous cells.

In an embodiment herein, the payload solution may be applied to the polymer solution and/or the formed anisotropic porous MNs before adding the substrate layer. If the anisotropic porous MNs are already formed then the payload may then be drawn to and/or distributed throughout the MN tips via, for example, capillary action. If the payload solution is added before formation of the anisotropic porous MNs, such as, for example, before the freezing stage when there is a pre-polymer solution, then the payload solution may mix with the pre-polymer solution. Without intending to be limited by theory, it is believed that such a process may increase payload loading efficiency and/or reduce payload waste in the substrate layer.

In an embodiment of the manufacturing method herein, a loading step occurs when the payload (e.g., in the payload solution) is loaded into the anisotropic porous MN prior to removing the anisotropic porous MN from the mold. In an embodiment herein, the loading step is after the lyophilization step. In such an embodiment, the loading step may occur via, for example, dipping the already-lyophilized anisotropic porous MN (or the MN array, etc. containing the anisotropic porous MN) into the payload solution, spraying the payload solution onto the mold containing the anisotropic porous MN, and a combination thereof. Without intending to be limited by theory, it is believed that even if the payload solution is added to the substrate layer, the channels will direct the payload to the channels in the tips of the anisotropic porous MNs, so that they can be effectively and uniformly-delivered.

In an embodiment herein, the loading step is after the anisotropic porous MN is removed from the mold. For example, after lyophilization the anisotropic porous MN is removed from the mold and contacted with a payload solution containing the payload. The payload solution is drawn into the anisotropic porous MN via capillary action.

In an embodiment herein, after the loading step, the anisotropic porous MN may be removed from the mold.

In an embodiment herein, the anisotropic porous MN, the MN array, the monitoring system, and/or the fluid sampling system; is manufactured without a centrifugation step while the optional payload is contained therein; or without a step where the mold is centrifuged while containing the payload. Without intending to be limited by theory, it is believed that, as compared to previously-known methods, such a centrifugation step is not needed to fill the channels with the payload, as the channels are not a mixture of closed and open cells as in previous cryoMNs. The advantages of avoiding a centrifugation step may include avoiding cell damage, allowing a broader diversity of potential payloads and a homogenous distribution and consistent loading of drugs, reduced manufacturing complexity, reduced technical expertise required to load the anisotropic porous MN, etc. As cells and proteins can be especially damaged by high-shear and/or high-centrifugal forces, the lack of such a centrifugation step when loaded with a payload is especially useful when the payload contains a cell, a macromolecule, and a combination thereof.

The cooling element herein may be, for example, a highly-refrigerated cooling plate, a container of ultra-low temperature liquid such as, for example, liquid nitrogen (b.p. −196° C.), liquid helium (b.p., −269° C.), dry ice (m.p., −78.5° C.), and mixtures thereof. In an embodiment herein, the cooling element is in the form of a cooling bath.

### 4. Examples

### 4.1 Example 1

In an embodiment herein, the pre-polymer solution contains 5% w/v gelatin (Sigma-Aldrich, Saint Louis, Missouri, USA), 1M 1-(3-Dimethylaminopropyl)-3-ethylcarbodiimide hydrochloride (EDC) (Energy Chemical, Shanghai, China), and 1M N-hydroxysuccinimide (NHS) (Macklin, Shanghai, China,) in an aqueous solution.

A PDMS negative mold as seen in FIG. 4 is provided containing a mold top surface and a mold bottom opposite the mold top surface. The mold bottom is open to the air. The mold contains the wells which narrow pointing towards the mold top surface to form the MN tips.

The pre-polymer solution is casted into the mold by adding it to the mold top surface and allowing the pre-polymer solution to fill the voids by centrifugation at a speed of 4,000 rpm. Excess pre-polymer solution is added to the mold so as to form the backing layer. Tin foil is then added to the mold bottom to cover the wells and form the backing layer.

The room temperature is 25° C. The mold bottom is placed onto a cooling plate cooled to a temperature of −20° C., so as to provide a temperature gradient of 45° C. for 10 minutes. After 10 minutes the pre-polymer solution is frozen solid and the mold including the frozen pre-polymer solution is stored in a −20° C. freezer for two days. The EDC and NHS react to form a polymeric matrix. The polymeric matrix, still in the mold, is then thawed in deionized water for 1 day to remove the unreacted chemicals.

The mold containing the thawed polymeric matrix is placed in a lyophilizer (Ningbo Scientz Biotechnology CO., LTD, SCIENTZ-10N, Ningbo, China) cooled to −80° C. to totally pre-freeze for 2 hours. The lyophilizer is sealed and the air evacuated, and the lyophilization process initiated. The process is completed in 4 hours resulting in a MN array containing the anisotropic porous MNs within the PDMS mold.

The mold containing the anisotropic porous MNs is removed from the lyophilizer and stored in a dry box at room temperature. The MN array contains a 10×10 array of anisotropic porous MNs, each MN having a needle height of about 850 m, a square base with each side of the square being about 260 m, and a backing layer thickness of about 1 mm. The pore diameter is around 100 m as measured using an optical microscope and confirmed with SEM measurements. The MN array is peeled out of the mold by pulling on the backing layer, resulting in a MN array containing a plurality of anisotropic porous MNs.

### 4.2 Example 2

A MN array is manufactured according to the process described in Example 1, except that the MN array is not removed from the mold and is formed without a backing layer. A payload of T cells (isolated from peripheral blood mononuclear cells) suspended in 2.5 wt. % DMSO and 100 mM sucrose PBS cryoprotectant medium is prepared. 30 μL of the payload is added to the mold bottom to form a loaded MN array without centrifuging the payload. A handle is applied covering all the MN bases in lieu of a substrate layer. The whole device (mold, pre-polymer solution, handle, etc. as seen in FIG. 9) is then frozen in a standard gradient cryopreservation process (−1° C./min) such as using a gradient cooling box filled with isopropyl alcohol. After the whole device is frozen solid, the handle is pulled and the frozen MN array is released from the mold and ready to use.

The handle is held and the frozen MN array is pressed into the skin. The MN array penetrates the skin tissue to about a depth of 50˜850 μm. The handle is held against the skin for about 2 min to allow the ice to melt sufficiently. The handle is removed leaving the MN tips and their payload embedded in the skin tissue.

### 4.3 Example 3

A MN array (1×10) is manufactured according to the process described in Example 1. A three-electrode sensor is connected to the substrate layer by a dissolvable adhesive layer. The MN array is then touched to the conjunctival meniscus in the eye of a patient to extract a sample of tears. The device is then immersed in the biomarker recovery buffer containing 0.1% Triton-100 in a substrate solution (i.e., 5 mM K3[Fe(CN)6]/K4[Fe(CN)6] in 0.1 M KCl/PBS buffer) for IFN-γ recovery from MNs. The MN will detach from the sensor surface, which makes the IFN-γ recovery more sufficient. After 10 minutes of recovery, the sensor is connected to an electrochemical device to detect the electric signal.

### 5. Method of Use

In an embodiment herein, the MN and/or the MN array herein may be part of, or contained within, a monitoring system, such as for monitoring a biological marker. In an embodiment herein the biological marker is IFN-γ found in tears. IFN-γ is a biological marker which may indicate inflammation of the eye or the presence of an inflammatory eye disease.

FIG. 10 shows a schematic view of an embodiment of a method of use of the MN patch according to the present invention. In FIG. 10, an eye 60 containing tears 62 is shown. A monitoring system 64 contains a microneedle patch 10 in operable relation to an electrochemical sensor 66. When the MN patch 10 is in contact with the tear 62, the MN patch 10 extracts a sample of the tear 62, which then is provided to the electrochemical sensor 66 for analysis.

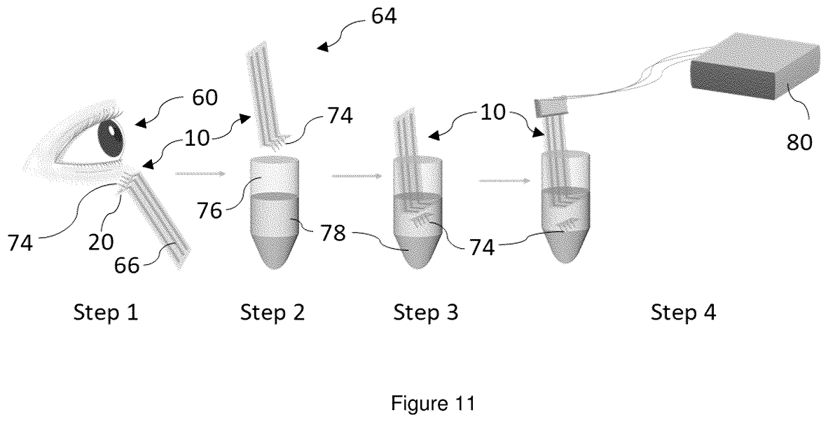

FIG. 11 shows a schematic view of an embodiment of a monitoring system 64 according to the present invention. In this case, the monitoring system 64, is a tear biological marker detection system, where Step 1 shows a MN array 10 on a horizontal substrate layer 20 extracting a biological marker 74 from tears from an eye 60.

Step 2 shows a reaction vessel 76, such as a test tube, containing a releasing solution 78 such as a biomarker recovery buffer which causes the MN array 10 to release any absorbed biological markers 74.

Step 3 shows the MN array 10, releasing the biological markers 74 into the releasing solution 78. Step 4 shows the MN array 10 connected to an electrochemical sensor, 80, which detects the biological markers 74 from the MN array 10 and/or the release of the biological markers 74 from the MN array 10 into the releasing solution 78.

In another embodiment herein, the biomarker is matrix metalloproteinase-9 (MMP-9) from tears as it is believed to be a possible biomarker for a variety of conditions, such as, for example, inflammation, dry eye disease, cancer.

In an embodiment herein, the anisotropic porous MN, a MN array, a monitoring system, etc. as described herein; or an unloaded anisotropic porous MN, an unloaded MN array, an unloaded monitoring system, etc., may be used to collect a sample, such as, for example, a bodily fluid, a liquid, and a combination thereof; or blood, urine, saliva, vaginal fluid, a tear, and a combination thereof; or a tear. Without intending to be limited by theory, it is believed that the tip surface of the anisotropic porous MN herein is so small, and the cross-linked polymeric matrix is so soft, that it avoids irritation to, for example, the eye surface. Therefore, using the anisotropic porous MN herein to retrieve microliter samples of, for example a tear directly from the eye reduces potential pain and irritation to the subject.

In an embodiment herein, the anisotropic porous MN, a MN array, a monitoring system, etc. as described herein; or an unloaded anisotropic porous MN, an unloaded MN array, an unloaded monitoring system, etc., may be used to deliver a payload, as described herein, to a target. The target may be, for example, a specific part of a human or animal, such as, for example the dermis, an eye, and/or other locations.

In an embodiment herein, the anisotropic porous MN is kept within the mold until immediately prior to use, so as to protect the structure of the anisotropic porous MN from damage, contamination, etc. during, for example, storage, transportation, etc.

### 6. Proof of Concept

Microneedles (MN) technologies are rapidly expanding in research and clinical translation. It is a potent and non-invasive delivery platform for a wide range of drugs, sensors, and target tissues. Their integration with advanced materials, electronics, and various controlling systems has resulted numerous applications in drug delivery, biosensing, and intelligent therapy systems.

Through the history of MN development, the precise control of its microstructure has been of great importance for designers. It determines the ultimate performance of the MN devices including mechanical properties and efficiency in drug delivery and body fluid sampling. Porous MNs have interconnected nano- or micron-sized pores through which fluids can be efficiently transported because of the channel-like connected pores by capillary action, thereby transporting the collected fluid to the analysis system or the drugs to the tissue. Compared with the non-polymer ones, polymeric porous MNs are usually biocompatible and biodegradable and can be easily fabricated without complicated micromachining processes as well as equipment and the requirement for a clean room environment.

It is noticed that existing porous MNs of the prior art only have randomly interconnected pores without the precise control of the pore diameter and running direction. In addition, due to the imperforated structure, these porous MNs cannot be used to deliver cell-based therapeutics.

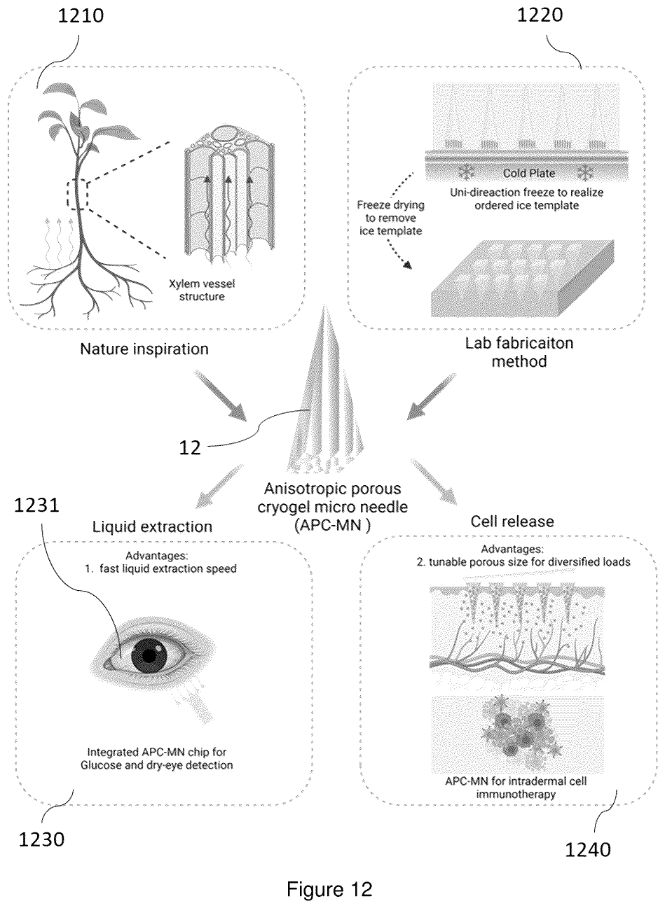

Referring now to FIG. 12, the inventors have discovered that incorporating an anisotropic porous structure, similar to xylem vessels 1210, in an MN device 12 enables highly efficient liquid transportation and delivery of drugs of varying sizes, from small molecules to cells, from the tip to the base.

At present, in the prior art, the porous structures are made by technologies such as emulsion followed by bonding, hot embossing, and pyrogen leaching. However, none of these technologies can achieve the goals of the present invention. Ice templating 1220, also known as freeze casting, is a technique that takes advantage of the highly anisotropic solidification behavior of a solvent, typically water, in a precursor solution followed by lyophilization, allowing for the controlled templating of a directionally porous material. This technique has been widely used in biomedical field due to its precisely controllable pore size, good shape recovery capacity, and irreversible compressive mechanical property.

The resulted MNs 12 with the anisotropic pores (APC-MNs) fabricated by ice templating 1220 are light in weight, flexible, and soft like bird feather. It shows good liquid absorption capacity compared to isotropic porous polymer MNs. It is also shown that the pore size can be precisely controlled by tuning the polymer concentration and freezing temperature. As the polymer concentration increases, the pore size decreases, whereas higher freezing temperatures result in an increase in pore size. This unidirectional porous structure and the feather-like texture make APC-MN 12 suitable for the extraction of tears 1230. Compared to commercial tear strips, APC-MN has smaller contact area with the ocular surface 1231, and the present finding indicates that APC-MNs 12 pose no risks to the ocular surface 1231. When it is used in the rats with the dry eye disease and diabetes, tear biomarkers are successfully recovered from APC-MNs, with the analyzed biomarker levels being consistent with those obtained from the strips.

Further, MNs are also used for cell delivery 1240. Briefly, γδ T cell suspension is absorbed directly into the APC-MN 12 due to the capillary force. The cell-loaded APC-MNs can be frozen to gain sufficient mechanical strength for skin penetration. The γδ T cell-containing device is then used to treat pleural mesothelioma and subcutaneous melanoma solid tumor mice models.

### 6.1 Fabrication and Characterization of the APC-MNs

Cryogelation is a process under semi-frozen conditions, leading to a polymer network cross-linked around ice crystals. Subsequent thawing/removing of ice crystals leaves behind a polymeric material with an interconnected network surrounded by a highly dense polymer wall. When the unidirectional freezing method is used in the process, the porous network will be oriented. If no temperature gradient is applied, the pores would be disordered.

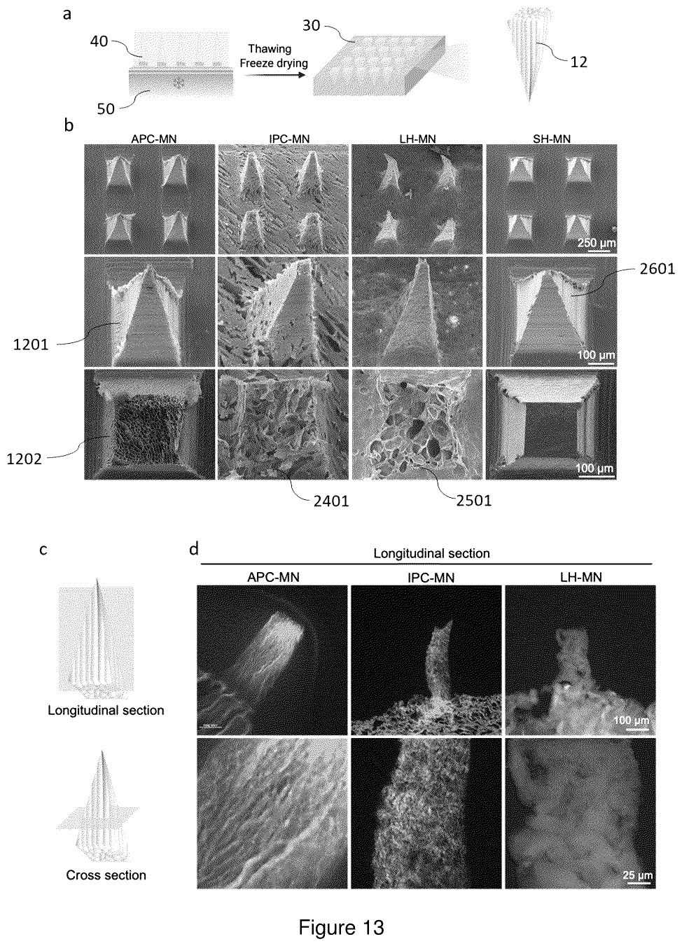

FIG. 13a shows an embodiment of a fabrication process of APC-MNs 12. Cryogelation with the unidirectional freezing is applied on the PDMS MN mold 30 containing a pre-polymer solution 40. This solution 40 was composed of polymer (e.g. gelatin), EDC and NHS and loaded into the tip holes of PDMS molds. Pure water was used to fill the base cavity of molds.

After a tin foil patch, which serves as a substrate layer of the APC-MNs 12, was covered on the mold 30, the sealed molds 30 were put on the cooling bath 50 with the tin foil downwards, in which the ice crystals growth oriented from the base of the molds. This gelation at −20 degrees lasted two days and APC-MNs were acquired through thawing to remove the unreacted molecules before lyophilization.

Three other types of MNs are also fabricated, they include isotropic porous cryogel (IPC), lyophilized hydrogel (LH), and solid hydrogel (SH) which serve as the controls. The IPC-MN was made similarly to APC-MN except for freezing under a uniform temperature environment instead of a temperature gradient. In contrast to the fabrication process of APC-MN and IPC-MN, which involved freezing, cryogelation, thawing, and lyophilization, LH-MN and SH-MN were prepared through the sequence of crosslinking, freezing, and lyophilization or drying at room temperature. The primary distinction lies in the sequence of crosslinking and ice crystal growth. When ice crystal growth happens first (APC-MN and IPC-MN), it leads to larger ice crystal growth resulting in an interconnected porous structure. On the other hand, when crosslinking is done prior to freezing, it limits the growth of ice crystals resulting in relatively isolated pores.

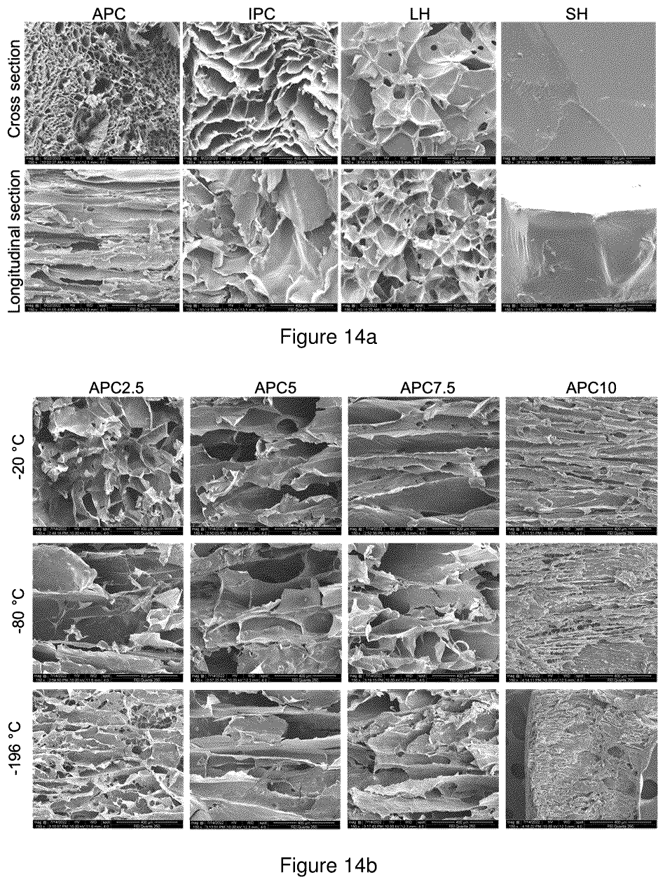

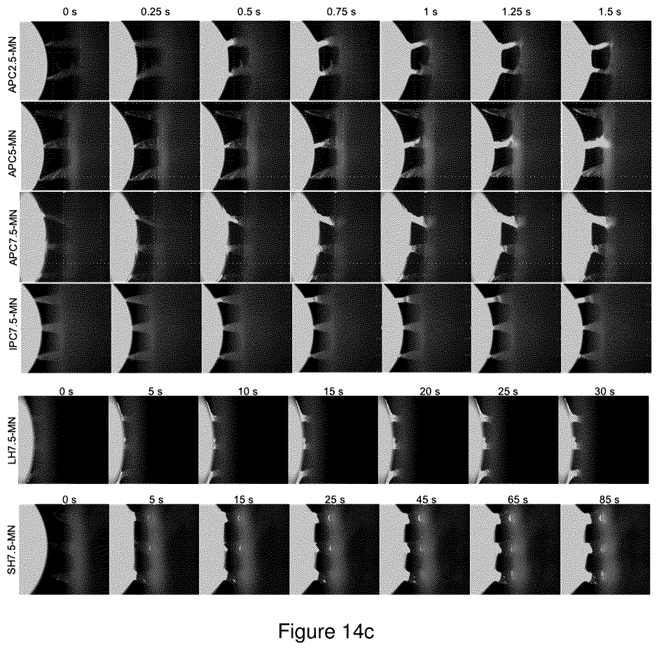

As shown in FIG. 13b, APC-MN 1201 looks the same as SH-MN 2601 from a top view. However, the cross-sectional view of APC-MN tips 1202 shows a lined porous structure. LH-MN 2501 has random and interconnected pores while IPC-MNs 2401 have random pores across the devices. These phenomena (different internal structures) were also seen when the mold was changed from MN morphology to the cylindrical shape, as shown in the SEM images of FIG. 14a. Referring to FIG. 13d, the porous structure of APC-, IPC-, and LH-MNs were further stained with Cy3 and the longitudinal section thereof is imaged by a fluorescence microscope after these MNs were frozenly sectioned.

The pore size of APC-MNs could be controlled by tuning the freezing temperature and polymer concentration. The SEM images in FIG. 14b demonstrate the APC-MNs with polymer concentrations of 2.5 wt. %, 5 wt. %, 7.5 wt. %, and 10 wt. % that were fabricated using freezing temperatures of −20° C., −80° C., and −196° C. It is found that the pore size decreased as the polymer concentration increased, but increased with a higher freezing temperature. Taken together, the anisotropic porous structure of MNs can be made by a unidirectional freezing method.

### 6.2 APC-MNs for Liquid Extraction

### 6.2.1 Liquid Extraction Capacity

Different APC-MNs with different pore size were made by adjusting polymer concentrations (2.5 wt. %, 5 wt. %, and 7.5 wt. %) for liquid extraction. The obtained APC-MNs were named as APC2.5-MNs, APC5-MNs, and APC7.5-MNs. The liquid extraction capacity of APC-MNs was tested by absorbing a drop of Cy3 dye solution. As shown in FIG. 14, all these APC-MNs can absorb liquid from tips to base within 1.5 s no matter what kind of pore size. With a specific polymer concentration (7.5 wt. %), APC-MNs showed the fastest liquid absorption speed in contrast to IPC-MNs, LH-MNs, and SH-MNs.

The APC-MNs displayed good liquid absorption performance due to the capillary force formed by the aligned channels. Although the fabrication process of APC-MNs and IPC-MNs is similar, the liquid absorption speed of APC-MNs was faster than that of IPC-MNs due to the anisotropic porous structure. LH-MNs have relatively isolated pores, while the liquid flow in SH-MNs depends on the hydrogel swelling, resulting in a much slower fluid absorption speed than that of APC-MNs.

### 6.2.2 APC-MNs for Tear Extraction

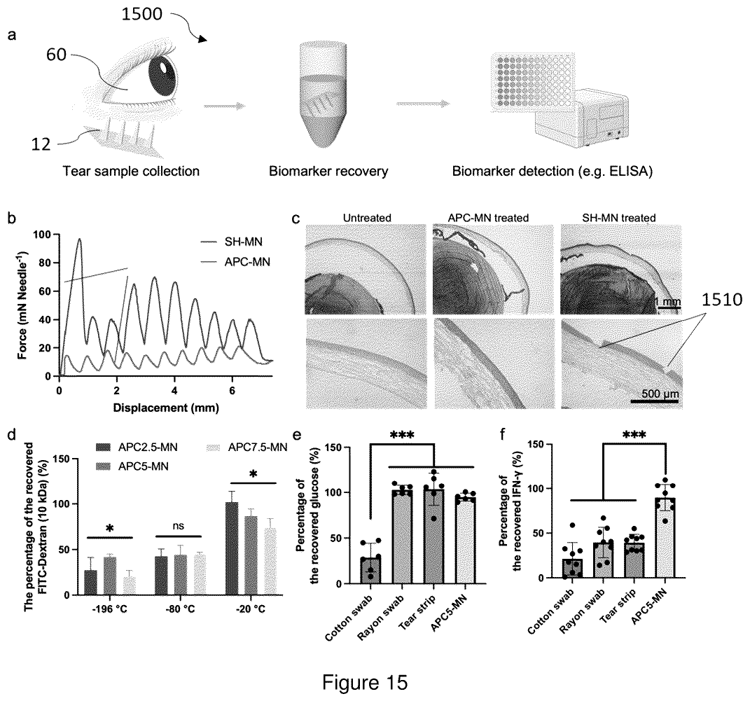

The excellent liquid absorption performance makes APC-MNs a good candidate for tear extraction. FIG. 15a shows an embodiment of a method of use 1500 according to the present invention, wherein the health of the eye 60 is monitored by using APC-MNs, including tear extraction, biomarker recovery, and post biomarker analysis.

To evaluate the risk assessment of APC-MNs used for eyes, the mechanical test and the mouse corneal layer damage test were performed.

FIGS. 15b and 15c show the mechanical performance of APC-MNs and SH-MN under a shear force. APC-MNs displayed significantly weaker mechanical property than SH-MN. After the application of shear force, both APC-MNs and SH-MN tips were bended, but APC-MNs bending required smaller shear force, indicating that the APC-MNs are soft and friendly to eye application.

Furthermore, to ensure that APC-MNs would not injure the eye corneal layer, APC-MNs were applied to the mouse eye, and SH-MN was employed as the control group. FIG. 15c shows that the H&E staining of untreated, APC-MNs treated, and SH-MN treated mouse corneal layers. No obvious damages are shown on the APC-MNs treated corneal layer which was similar to the untreated group. For SH-MN treated group, the obvious tip holes 1510 could be observed on the corneal layer. These data suggested that APC-MNs showed weaker mechanical performance than SH-MN and would not injure eyes.

The biomarker recovery test was performed in vitro to verify if the biomarker can be released from the MN tips. FITC-dextran (10 kDa) was used as a model biomarker to mimic the recovery of tear biomarker from APC-MNs fabricated with different polymer concentrations and different freezing temperature. The results of which are shown in FIG. 15d. The FITC-dextran recovery ratio of APC-MNs increased with the decrease of the freezing temperature or polymer concentration. APC5-MN fabricated with a freezing temperature of −20° C. was chosen as the tear extraction device due to its comparable biomarker recovery ratio with control groups as well as its better anisotropic porous structure than APC2.5-MN's.

Furthermore, the real disease biomarkers in tears, glucose as a diabetes biomarker and IFN-gamma as a dry eye biomarker, were used to release from APC-MN, tear strip (Surgi Edge, india), rayon swab (Huayue, China), and cotton swab (Wastons, Hong Kong SAR), as shown in FIGS. 15e and 15f. The glucose recovery rate of APC-MN was close to 100%, indicating the tear glucose can be totally release from the APC-MN. The recovered percentage of IFN-gamma from APC-MN was the highest compared to the commercialized tear strip and swabs, suggesting its potential for monitoring eye health.

### 6.3 APC-MNs for Drug Delivery

6.3.1 Drug Loading into APC-MNs

Different APC-MNs were made using gelatin concentrations of 2.5, 5, and 7.5 wt % and isotropic porous MN was made of gelatin concentrations of 5 wt %. They are named by APC2.5, APC5, APC7.5, and IPC5, respectively.

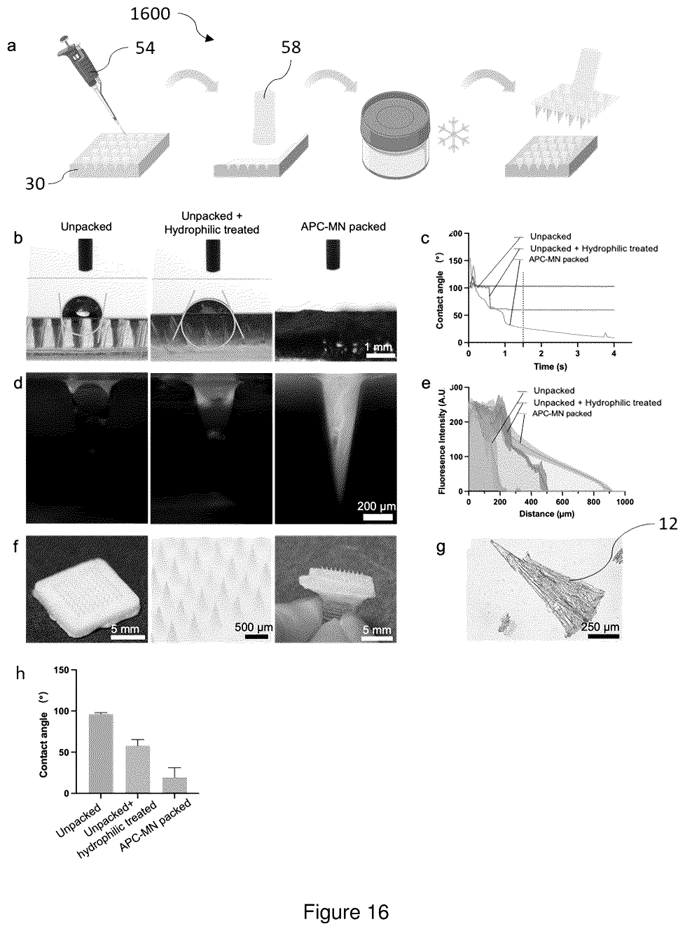

FIG. 16a shows a schematic view of an embodiment of the manufacturing method 1600 of an APC-CryoMN. Freeze-dried hydrogel (with gelatin concentrations of 5 wt %, FH5) was also used to make coated PDMS mold to compare the effect of FH5 and APC-MN on the liquid absorption ability. By using this ready to used APC-MN packed mold 30, only 10 μL of the desired liquid 54 was needed to fill the tips followed by adding supporting solution as base, greatly reducing the amount of liquid needed for the MN mold 30. After placing a holder or a handle 58 onto the molds and cooling them down, the APC-CryoMN 12 can be stored for a long time under low temperature such as liquid nitrogen. A well-shaped APC-CryoMN 12 was formed after demolding and ready to be used. Thus, the process of preparing cryoMNs with the APC-MNs was relatively simple and efficient, as no centrifugation or vacuum was needed.

To characterize the hydrophilic property of APC-MNs, a drop of water was added to PDMS negative molds including unpacked (untreated), unpacked but hydrophilic treated mold (plasma-treated), and APC-MN packed (APC-MN filled) molds, respectively, for measuring the water contact angle.

FIG. 16b shows optical images indicating water contact angles on a mold that is unpacked, unpacked but hydrophilic treated, and APC-MN packed. The water contact angle for the unpacked PDMS mold was 96.4°±1.6°, and around 58.2°±7.10 for the hydrophilic-treated mold after 1.5 s. On the other hand, as shown in FIG. 16c, the water contact angle of the APC-MN packed mold was 27.2°±5.0° in 1.5 s and still continuously decreased. A comparison of the water contact angles of the unpackaged, unpackaged but hydrophilic treated and APC-MN packed PDMS molds is shown in FIG. 16h. The large water contact angle indicated the hydrophobic nature of the unpacked molds, while the hydrophilic treated molds displayed improved hydrophilic property. As opposed to the unpacked and hydrophilic-treated molds, the APC-MN packed molds exhibited a hydrophilic property. Cy3 fluorescent dye solution was used to visualize the liquid distribution in the tip holes of APCMN packed molds.

As shown in FIG. 16d, compared to the unpacked and hydrophilic treated groups, the fluorescence of APC-MN packed groups filled the whole tip. The penetration distance of dye in the unpacked mold and hydrophilic treated mold were 223.4±50.9 μm and 486.4±25.5 μm, respectively, while the penetration distance in the APC-MN packed molds was 897.6±26.7 μm, as shown in FIG. 16e. Only in the APC-MN packed molds that liquid demonstrated the ability to completely fill the tip holes of the PDMS mold, indicating the excellent liquid absorption ability of the aligned porous gelatin APC-MN.

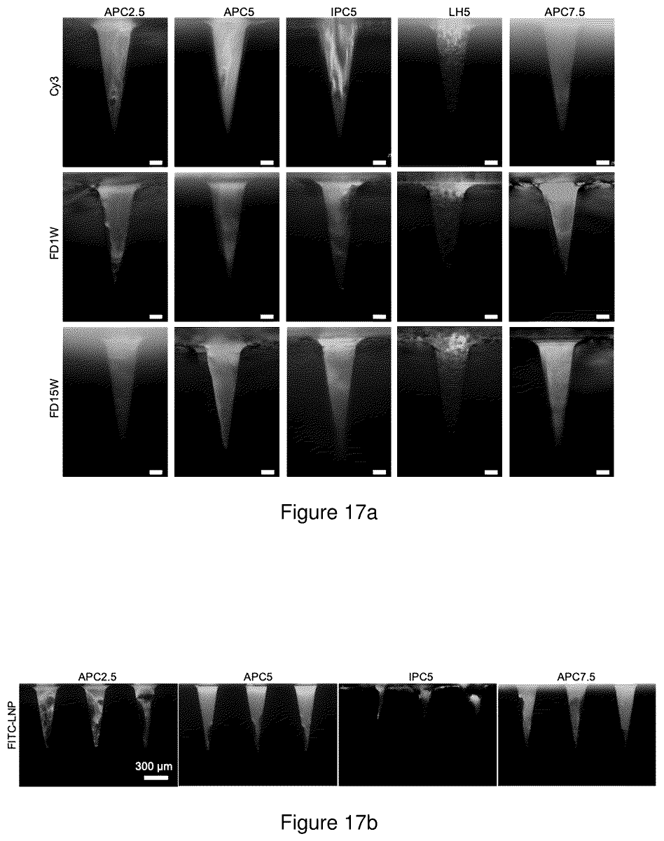

It is an important characteristic that various therapeutics can be absorbed into tip cavities of APC-MN. Cy3 and FITC-dextran with different molecular weights (10 kDa and 150 kDa) (FD1W and FD15W) were used to mimic small molecular and various macromolecular drugs. Compared to LH5-MNs, all APC2.5-, APC5-, APC7.5-, and IPC5-MNs can absorb the liquid into tip cavities regardless of the molecular weight. The distribution of fluorescence dye with different molecular weight (Cy3 and FITC-dextrans with 10 kDa and 150 kDa molecular weights or FD1W and FD15W) in the tip of APC2.5-, APC5-, IPC5-, LH5-, and APC7.5-MNs is demonstrated in FIG. 17a, wherein the scale bar=100 μm. Benefiting from the interconnected macroporous structure of APC-MNs and IPC5-MN, liquid can penetrate and fill the whole tip hole, no matter the pore size or orientation. Nevertheless, the small and isolated pores in the tip of FH-MN packed mold prevent the liquid from filling completely, leading to the trapping of the solution in a shallow layer. FITC encapsulated lipid nanoparticles (FITC-LNP) were also used to add into APC2.5, APC5, APC7.5, and IPC5-MNs. With reference to FIG. 17b, the fluorescence of FITC-LNP distributed in the tips of APC2.5-, APC5-, and APC7.5-MNs, but little FITC-LNP entered the tips of IPC5-MNs.

### 6.3.2 APC-MNs for Cell Delivery

Next, the construction of APC-cryoMN was tested with γδ T cells. PBS with 2.5% (v/v) DMSO and 100 mM sucrose mixture was used as the cryogenic medium (CGM) to cryopreserve γδ T cells in cryoMNs.

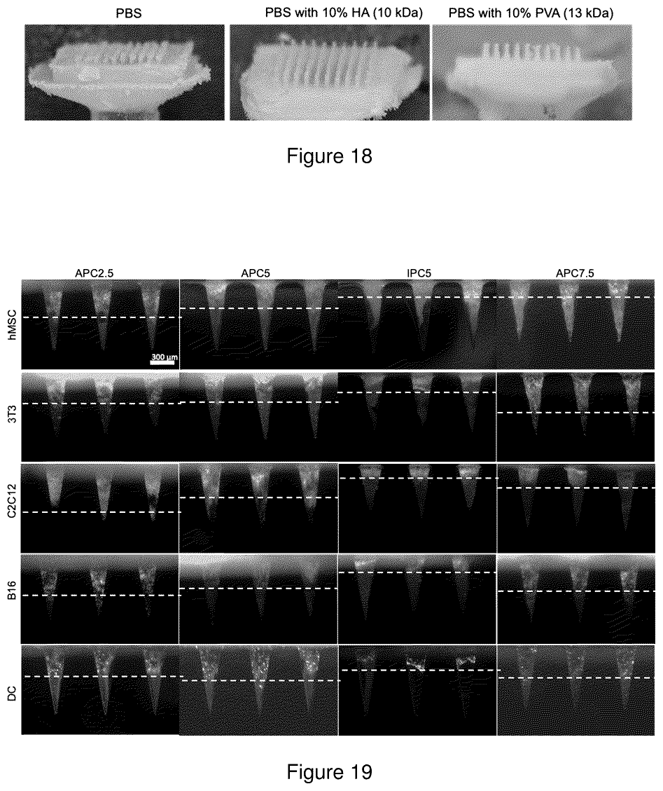

To verify the feasibility of the fabrication process, the CGM was firstly added to fill the tip of APC-MNs, and supporting solution was subsequently dropped to fill the base part. After freezing for 24 hours, the APC-cryoMN tips were broken during the demolding process when the supporting solution was pure PBS buffer. However, APC-cryoMNs can be well-demolded from the PDMS mold when the base consisted of polymer solution like PBS with 10% HA and PBS with 10% PVA. FIG. 18 displays photographic images of demolded APC-cryoMNs with different base formulations, namely PBS, PBS containing 10 wt. % 10 kDa HA, and PBS with 10 wt. % 13 kDa PVA. PBS with 10% PVA was finally selected as the base solution because PVA is cheaper than HA and have been reported as a cryopreservation agent.

Referring back to FIG. 16f, the MN tips were well shaped when PBS with 2.5% (v/v) DMSO and 100 mM sucrose was used as tips and PBS with 10% PVA was used as MN base. A 3D printed holder embedded in supporting solution was used to prevent APC-cryoMN being melted with the heat from fingers. After melting in PBS, both the CGM of tips and base dissolved immediately, and MN tips with aligned porous structure detached from the holder. A close-up image of APC-CryoMN after melting in PBS is shown in FIG. 16g, it can be seen that the microneedle 12 possess an aligned porous structure.

### 6.3.3 Characterization of APC-cryoMNs

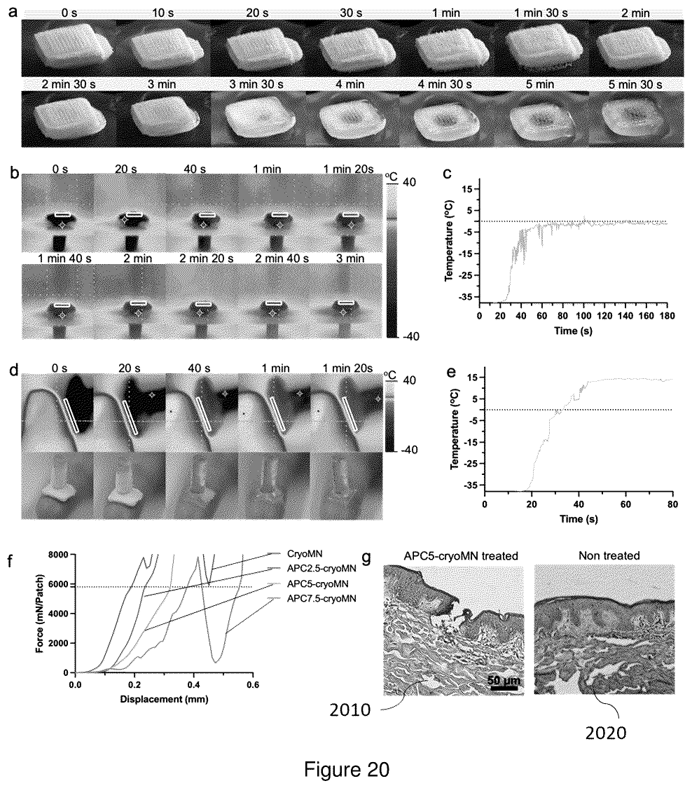

As the application's target site is human skin, ice crystals would melt at both room temperature (RT) and contact with the skin. The evaluation of the thawing temperature of APC-cryoMN is critically important to help us understand the suitable time to use it. As shown in FIG. 20a, after removing APC-cryoMN from liquid nitrogen storage environment (−196° C.), frost formed on tips immediately and gradually increased under RT at 21° C., and the phase transition of ice to water occurred after 1 minute.

Thermal imaging was applied to record the temperature change of APC-CryoMNs during the thawing process. Due to the limited detection of the temperature range of the equipment, the temperature change was recorded when temperatures were above −40° C. As shown in FIGS. 20b and 20d, the color of selected tip area (white box) changed from dark blue to blue, indicating the increasing temperature during thawing process under RT (FIG. 20b) and finger skin temperature (FIG. 20d).

FIGS. 20c and 20e demonstrate the quantified temperature is quantified to change profile of tips to analyze the lifetime of APC-cryoMN under RT (FIG. 20c) and skin temperature (FIG. 20e). Within 13.2 s, 29.2 s, and 31.5 s, the tip temperature rose from −196° C. to −40° C., −30° C. and −20° C., respectively (FIG. 20c). After approximately 1 min, the temperature approached 0° C. When touched with fingers, the temperature of the APC-cryoMN tips increased to about −40° C., −30° C., −20° C., and 0° C. after approximately 10.9 s, 20.6 s, 23.5 s, and 33.1 s, respectively (FIG. 20d). The temperature approached to 15° C. after 55.2 s. These findings suggest that the ideal residence time for the APC-cryoMN at RT should be within 29 s.

This aligns with the previous research that the cryoMNs can penetrate porcine skin within 30 s without any significant differences in penetration depth. Moreover, during the cell thawing process, 3.5% ice crystal formed range from −40° C. to −30° C., while 75% ice crystal forms between −10° C. and 10° C. To minimize the ice crystal damage to the cells, the APC-cryoMNs should be applied within 29 s after removing from liquid nitrogen.

To ensure the skin penetration ability of APC-cryoMNs, the mechanical properties were measured by the compression test and porcine skin insertion. Generally, the skin insertion process takes a few seconds. Thus, a compression speed of 0.5 mm/s was set to conduct the mechanical test.

As shown in FIG. 20f, the cryoMNs and APC-cryoMNs except APC-cryoMNs (with 7.5 wt % gelatin cryogel) can bear the compressive force of 5.8 N that has been reported as the force for skin penetration. With the increase of polymer concentration, the compression forces that APC-cryoMNs can bear decreased, indicating the weak mechanical property when the higher concentration polymer was used to fabricate APC-MN packed molds. This is likely because the pore size becomes smaller with increasing polymer concentration, preventing the ice crystals from growing during the freezing process. APC5-cryoMN was applied on the porcine skin, and as shown in FIG. 20g, there was apparent damage of stratum corneum layer 2010 compared to non-treated skin 2020, demonstrating the ample skin penetration ability of APC5-cryoMN.

### 6.3.4 In Vitro Test of Cell Loaded APC-cryoMN

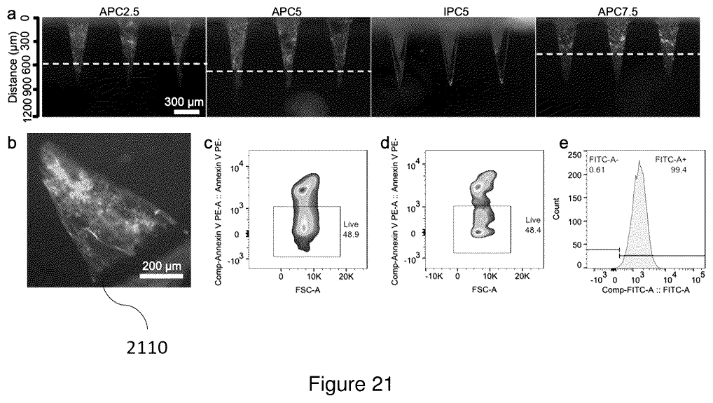

Cell loaded APC-cryoMNs are made by dropping cell suspension on APC-MN packed molds followed by gradient freezing. Six types of cells (hMSC, C2C12, B16, 3T3, dendritic cell, and γδ T cell) were loaded to evaluate the cell penetration ability of APC-MN including (APC2.5-, APC5-, and APC7.5-MN), with reference to FIG. 19 and FIG. 21a. IPC5-MN was taken as the control, where few cells could get into the tips. In contrast, all six types of cells can easily get into the tips of APC-MNs. Similar to the distribution of LNP, less cells penetrate into the tips of IPC5-MN packed mold, while cells can be absorbed into tip cavities of all anisotropic porous structure, demonstrating that the unidirectional porous structure can improve the cell loading performance. FIG. 19 demonstrates the cell penetration of hMSC, DC, 3T3, B16, and C2C12 in different APC-MN packed PDMS molds.

Moreover, for the cells with larger size ranging from 15 to 30 μm, including hMSC, C2C12, 3T3, and B16, APC2.5-MN packed mold show the deeper penetration distance in contrast to APC5 and APC 7.5-MN packed mold. Nevertheless, for dendritic cell (cell size: 10 to 15 μm) and γδ T cell (cell size: 5 to 7 μm), 8, 9 cells reached a greater depth of absorption within the tip holes of the APC5-MN packed mold compared to the other two APC-MN packed molds. In the case of the APC5-MN packed mold, smaller cells resulted in larger penetration depth. These results demonstrated that the adjustable pore size of APC-MN allows for adaptation to different types of cells. Based on these results, APC5 porous MNs were selected for loading γδ T cells into cryoMN.

Since the base part of APC-cryoMN was made of PVA solution, the tips detached from the MN base once the APC-cryoMN melted.

As shown in FIG. 21b, γδ T cell loaded tips 2110 detached when the APC-cryoMNs were taken from liquid nitrogen and placed in pre-warmed PBS. Cell viability of γδ T cell cryopreserved with the cryogenic medium (PBS with 2.5% (v/v) DMSO and 100 mM sucrose) was tested in both cryotubes and APC-cryoMNs. Results of which are shown in FIGS. 21c and 21d. Dead cells stained with ethidium homodimer-1 could be detected by flow cytometry due to the emitted fluorescence, while rest of cells do not show any signals. The cell viability of γδ T cells in vial tubes and APC-cryoMNs was 48.9% and 48.4%, respectively, indicating APC-MN packed molds do not have any negative effect on the live cell percentage after cell thawing. Furthermore, to ensure the cell functionality such as recognize and kill tumor cells after APC-cryoMN insertion, the surface biomarker of γδ cells was detected after cell thawing. 98.8±1.1% of γδ T cells still retained the expression of Vδ2-TCR (T cell receptor) after cryopreserving for one month, as shown in FIG. 21e. The percentage of Vδ2+ cell was similar to that of primary isolated Vδ2+ cells, suggesting that the surface biomarker would not change after cryopreserving in APC-MNs.

### 6.3.5 Delivery of γδ T Cells by APC-cryoMN for Mesothelioma ACT In Vivo

Mesothelioma is a type of aggressive solid tumor mainly caused by exposure to asbestos fiber. It originates from the mesothelium, which is a thin layer of tissue that covers many internal organs, and the tumor can spread across it.

With the lack of accurate and reliable biomarkers for detection, mesothelioma is often diagnosed at the late stage with limited treatment options. With the advances in CAR T therapy, mesothelioma remains difficult to treat due to the lack of a specific antigen acting as the target. γδ T cell has been studied as a potential therapeutic cell to against mesothelioma due to its tumoricidal activity without the need to recognize a specific tumor antigen. The cytotoxicity of γδ T cells against mesothelioma cells was reported previously, which could induce pyroptosis via the canonical pathway with efficacy in a tumor xenograft mice model.

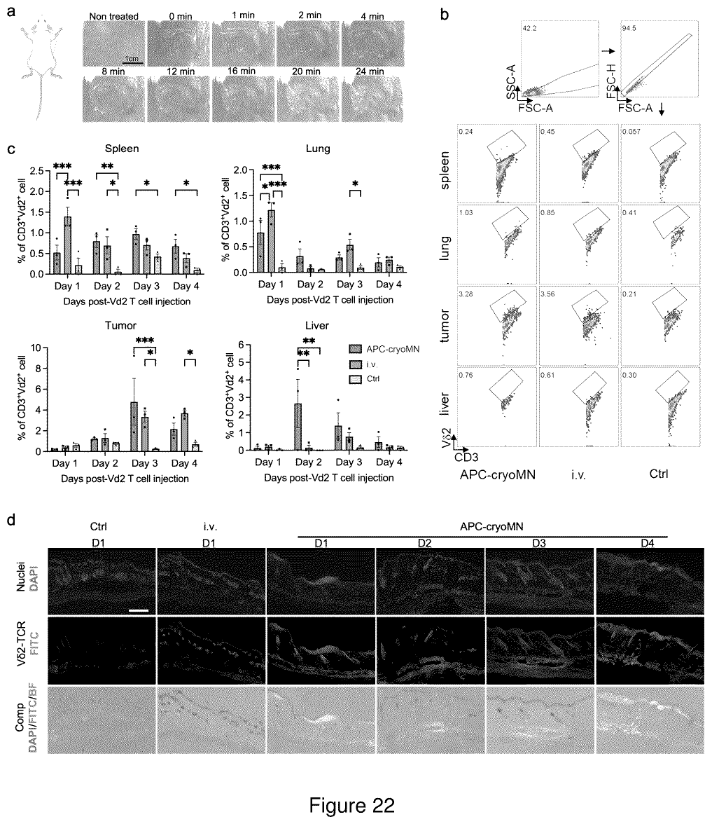

Based on the findings, the APC-cryoMN was utilized to load the γδ T cells for ACT delivery against intrapleural mesothelioma in vivo, results of which are shown in FIG. 22. The γδ T cells loaded APC-cryoMNs were applied on the posterior skin of tumor-bearing mice by pressing the holders onto them. To make sure the tips stay in the skin layers, the holder was removed after 1 min when the APC-cryoMN fully melted. The tip holes were apparent after removing the holder immediately, and they disappeared after 12 min.

To test whether Vδ2 T cells could infiltrate into the tumor, Vδ2 T cells were administered by i.v. and APC-cryoMN on 3 days post tumor injection in the MSTO-luc bearing mice. Total 1×107 of Vδ2 T cells were used for i.v. injection. According to the cell viability of cryopreserved γδ T after thawing, 2×107 of Vδ2 T cells were used for APC-cryoMNs to maintain the number of viable cells injected when compared to i.v. MSTO-luc mice were sacrificed on day 1, 2, 3, and 4 post-transfer. The spleen, lung, liver, and tumor were extracted to examine the presence of Vδ2 T cells by flow cytometry. In both the iv. group and APC-cryoMN group, CD3⁺Vδ2⁺ cells were detected in the spleen and lungs on the first day after Vδ2 T cell injection. However, the detection of Vδ2 T cells in the spleen tends to decrease from day 1 to day 4 post Vδ2 T cells injection in both groups. Similarly, the detection of Vδ2 T cells in the lungs, when using intravenous injection, also decreased from day 1 to day 4.

In contrast, when using APC-cryoMN, the detection of Vδ2 T cells in the lung initially increased from day 1 to day 3, followed by a decrease on day 4. In the liver, the detection of Vδ2 T cells remained at a low level throughout the 4-day period in the i.v. injection group, while it increased on day 2 and decreased on day 3 when using APC-cryoMN. Importantly, the detection of Vδ2 T cells in the tumor increased from day 1 to day 4 post Vδ2 T cells injection in both groups, as referenced in FIG. 19.

It is noted that in FIG. 22, the scale bar is 200 μm. Data represents mean±SEM from 3 independent experiments. Two-way ANOVA statistical test was used. *P<0.05, **P<0.01, ***P<0.001.

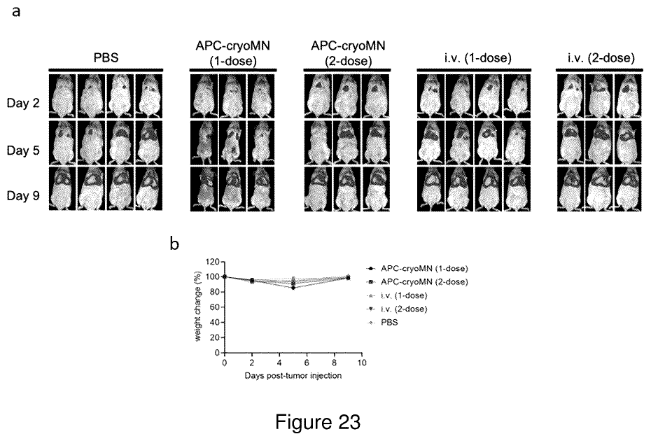

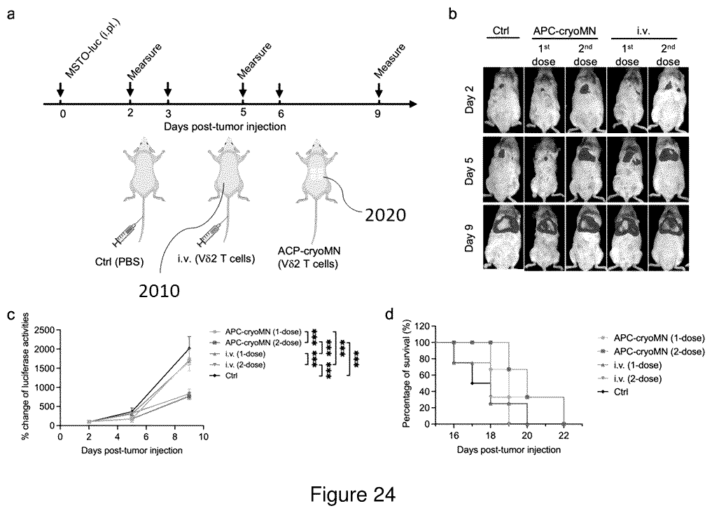

Next, it is sought to compare the efficacy of Vδ2 T cells administered via i.v 2010 and APC-cryoMN 2020 delivery for mice with mesothelioma. As shown in the timeline of FIG. 24a, one or two doses of Vδ2 T cells were applied on day 3 and/or day 6. Evaluation of tumor luciferase activity revealed that a single dose of Vδ2 T cells administered via i.v. injection and APC-cryoMNs resulted in ˜16.4% and ˜14.7% reduction in tumor growth, respectively, as indicated by FIGS. 24b and 24c. Furthermore, as shown in FIG. 24d, the administration of two doses of Vδ2 T cells via i.v. injection 2010 and APC-cryoMNs 2020 led to a greater reduction in tumor growth by ˜58.6% and ˜62%, respectively, compared to PBS control. The detection of luciferase activity of MSTO-luc mice in each treatment group over the course of experiment is shown in FIG. 23a, wherein each group consisted of three to four mice. FIG. 23b illustrates the weight changes of MSTO-luc mouse groups.

Based on these results, APC-cryoMN delivered Vδ2 T cells has similar efficacy to i.v. injected Vδ2 T cells in targeting mesothelioma, with a slight increase in the mice survival by two more days, as is also shown in FIG. 24d.

This has important implications as APC-cryoMN-delivered Vδ2 T cells could transverse the dermis into the bloodstream but could still infiltrate into the different organs and tumor mass at efficiency comparable to direct i.v. delivery to the blood circulation. With the non-invasiveness of APC-cryoMN, it may pose as an important tool to be developed for ACT in the future.

It is noted that in FIG. 24, each of the treatment groups contains 3 to 4 mice. Data represents mean±SEM. Two-way ANOVA statistical test was used for C. ***P<0.001.

### 6.3.6 APC-cryoMN Delivered Vδ2 T Cells for Melanoma Treatment In Vivo

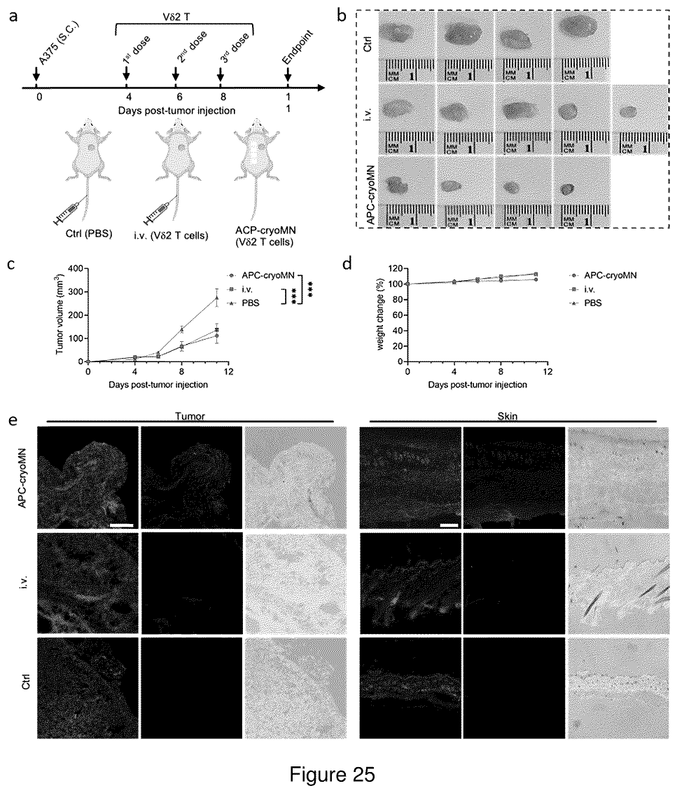

Based on the above findings, the effectiveness of Vδ2 T cells administered via APC-cryoMNs in targeting melanoma tumor is further investigated in another xenograft mice model, as is shown in FIG. 25a. Solid tumors were formed through subcutaneous (s.c.) injection of A375 cells for four days. Vδ2 T cells were delivered on day 4, 6, and 8 post-tumor injection, by i.v. injection and APC-cryoMNs, respectively.

FIG. 25b shows photographic representations of the tumor volume of mice receiving PBS as control, Vδ2 T from i.v., and APC-cryoMN routes over a period of 11 days; and FIG. 25c shows a graph indicating the tumor volume extracted from a mice from each experimental group over time. The application of Vδ2 T cells via i.v. injection and APC-cryoMNs resulted in a reduction of tumor growth by ˜50.2% and ˜59.4%, respectively, compared to the PBS control. No significant weight loss in the mice was observed throughout the experimental period, as demonstrated in FIGS. 25d and 25e.

With respect to FIG. 25, the scale bar is 200 μm, and the data represents mean±SEM. Two-way ANOVA statistical test was used for B. ***P<0.001.

### 7. Further Details

### 7.1 Experimental Section

### Fabrication of APC-MNs

PDMS MN molds were made by applying a mixture of PDMS prepolymer and curing agent (mass ratio: 10:1) onto a stainless-steel MN patch (10×10) (Micropoint Technologies, Singapore) through casting. The molds were then degassed in a vacuum oven and cured at 70° C. for 2 hours. Subsequently, the PDMS negative MN molds were obtained by demolding them from the stainless-steel MN patch. The obtained PDMS molds were treated with ultrasonic cleaning in 75% ethanol for 30 min and sterilized by ultraviolet exposure for 1 h before packing APC-MN inside.

To fill the MN cavities, a 50 μL volume of the prepolymer solution containing gelatin and EDC/NHS was firstly sterilized by a 0.22 μm syringe filter (Mlllex®-GP Filter Unit, Merck), and then casted into the PDMS molds and subsequently centrifuged at 4,000 rpm for 3 minutes. After centrifuging, 40 μL of solution was removed, and 150 μL of deionized water was added to fill the base cavity of the mold, followed by covering aluminium foil on top. The PDMS molds were placed on a cooling bath with the aluminium foil side facing downwards and rapidly cooled to −20° C. from the bottom, while the top side remained at room temperature. The molds were then stored at −20° C. for 48 h to allow for the crosslinking of the gelatin cryogel. Following this, a dialysis process was carried out overnight at room temperature using deionized water to remove the ice base and any unreacted chemicals in tips. The fabrication process took place in a sterile environment of a biological safety cabinet (BSC, Thermo Fisher Scientific). Subsequently, the PDMS molds were freeze-dried at −80° C. for 4 h, resulting in the APC-MN packed MNs. The obtained APC-MN packed MNs were sealed in a sterile and dry environment for the subsequently cell loading. The anisotropic porous cryogel MNs with different gelatin concentrations were fabricated and packed into the PDMS molds, with the same name as the samples in the last chapter, APC2.5-MN, APC5-MN, and APC7.5-MN respectively. The IPC-MN packed molds were prepared similar to APC-MN packed molds but frozen at a −20° C. freezer without aluminium foil covered. To create LH-MN packed molds, the negative MN mold was filled with the same gelatin prepolymer solution. After filling, the solution in the base cavity was removed, and the mold was frozen after the complete crosslinking of the gelatin hydrogel in the tips at room temperature. Finally, the molds were lyophilized.

### 7.2 Hydrophilic Property Test

The water contact angle measurements were performed on the surfaces of APC-MN packed mold by means of sessile drop technique using an OCA15EC drop tensiometer (DataPhysics Instruments, Filderstadt, Germany). For contact angle measurements, a water drop of 10 μL was deposited on the surface and the drop profile was recorded to determine the contact angle at the water/solid/air interface.

### 7.3 Cell Culture

Human mesenchymal stem cells (hMSCs), mouse melanoma cell line (B16), mouse myoblast cell line (C2C12), mouse fibroblast cell line NIH/3T3 (3T3), and human melanoma cell line (A375), were cultured with high-glucose DMEM supplemented with 10% FBS and 1% P/S in culture dish at 37° C. and 5% CO₂. Mouse bone marrow-derived dendritic cells (DCs) were cultured in complete RPMI 1640 medium supplemented with GM-CSF and IL-4 at 37° C. and 5% CO₂. Human γδ T cells, and luciferase expressing human mesothelioma cell line (MSTO-luc) was cultured with RPMI 1640 medium (ATCC modification) supplemented with 10% FBS and 1% P/S in culture dish at 37° C. and 5% CO₂. The culture medium was changed every 2 or 3 days, and a culture dish with 80 90% confluence for adherent cells was used for further cell experiments.

### 7.4 Drug and Cell Penetration Test

Cy3 was used to evaluate the drug penetration capacity as a small molecular drug model. 10 μL of Cy3 solution was added on the APC2.5-MN, APC5-MN, and APC7.5-MN packed single array molds (1×10). 10 kDa FITC-Dextran and 150 kDa FITC-Dextran were applied in the same way as macromolecular drug models. IPC-MN and LH-MN packed molds were compared with APC-MN packed molds. 10 μL of FITC encapsulated lipid nanoparticles (FITC-LNPs) were added on the APC2.5-MN, APC5-MN, and APC7.5-MN packed molds, which were used as the matrix/formulation drug model. IPC-MN packed molds were compared with APC-MN packed molds. Six cell types with the cell density of 1×105 cell/mL were used to evaluate the cell penetration capacity of APC-MN packed molds, including human mesenchymal stem cells (hMSCs), human γδ T cells, mouse melanoma cell line (B116), mouse dendritic cells (DCs), mouse myoblast cell line (C2C12), mouse fibroblast cell line NIH/3T3 (3T3). The distribution of Cy3, 10 kDa FITC-Dextran, 150 kDa FITC-Dextran, FITC-LNPs, and six type cells in tips was observed by a fluorescence microscope (Ti2-A, Nikon)

### 7.5 Fabrication of the APC-cryoMN

The APC-cryoMN was prepared by directly adding 10 μL cryogenic medium of PBS with 2.5% (v/v) DMSO and 100 mM sucrose mixture on APC2.5-MN, APC5-MN, and APC7.5-MN packed molds to fill the tip cavities. 120 μL of PBS, PBS with 10% (w/v) HA, or PBS with 10% (w/v) PVA was added to fill the base cavities. After that, the sample was frozen in the cryopreservation box frozen at −80° C. for 24 h and finally stored in liquid nitrogen. The obtained APC-MNs were named as APC2.5-cryoMN, APC5-cryoMN, and APC7.5-cryoMN respectively.

### 7.6 Mechanical Test of APC-cryoMN

Instron Tensile Tester 5942 was used to perform mechanical testing for APC2.5-cryoMN, APC5-cryoMN, and APC7.5-cryoMN. The APC-cryoMNs used in this test were prepared with 10% (w/v) PVA in PBS solution to fill up the base. After demolding, the APC-cryoMNs were preserved in liquid nitrogen until use. The two force load cells were precooled with liquid nitrogen followed by placing the APC-cryoMNs with tips upwards. The compression test was set up with a compressive speed of 0.5 mm/s. Empty PDMS mold filled with PBS with 2.5% (v/v) DMSO and 100 mM sucrose in tips and PBS with 10% (w/v) PVA in base was used as the control group, namely cryoMN.

To verify the mechanical property for skin penetration, APC-cryoMNs were further inserted into porcine cadaver skin obtained from a local supermarket. The porcine skin insertion was done by applying thumb pressure. The penetrated porcine skin tissue was fixed with 4% paraformaldehyde and then stained with H&E according to the protocol provided by the manufacture for histological analysis.

### 7.7 Melting Process

The melting process of APC-cryoMNs was recorded after taking APC-cryoMN from liquid nitrogen by an infrared thermal imager (FLIR ONE) at both room temperature and body temperature (finger skin).

### 7.8 Purification and Expansion of γδ T Cells

Lymphoprep density gradient medium was used to isolate peripheral blood mononuclear cells (PBMC) from human blood obtained from Hong Kong Red Cross. Tetrakis-pivaloyloxymethyl 2-(thiazole-2-ylamino) ethylidene-1,1-bisphosphonate (PTA) was used to stimulate the expansion of PBMC in recombinant human IL-2 protein. The PBMC was cultured with RPMI 1640 medium and 10% FBS, and incubated at 37° C. under 5% CO₂ for a total of 13 days. 50% media change was performed every 2-3 days during the 13-day period. Afterwards, γδ T cells were purified using human TCR γ/δ+ T Cell Isolation Kit to achieve >95% purity of CD3+Vδ2+ cells.

### 7.9 Fabrication of Cell-Loaded APC-cryoMNs

γδ T cells were suspended in cryogenic medium (PBS supplemented with 2.5 v/v % DMSO and 100 mM sucrose) at the cell density of 5×10⁸ cells/mL. Next, 10 μL of cell suspension (5×10⁶ cells) was added into the APC-MNs that were still inside the PDMS mold. 120 μL of 10% (w/v) PVA solution was then added to each mold to fill up the base, and a pre-sterilized holder was placed on top. The molds were placed at −80° C. for 24 h and then stored in liquid nitrogen. The APC-cryoMNs were demolding before use.

### 7.10 Visualization of γδ T Cell Loaded Tips after APC-cryoMNs Thawing

The detached cell-loaded APC-CryoMN tips were visualized after thawing. Specifically, yb T cells were firstly stained by the CellTracker™ Green CMFDA reagent according to the manufacture's protocol. 10 μL of stained γδ T cells at the density of 5×10⁸ cells/mL were cryopreserved in APC-cryoMN in a −80° C. freezer for two days. After that, the cell loaded APC-cryoMN was place into prewarmed PBS immediately. The tips loaded with fluorescent γδ T cell were then imaged by a fluorescence microscope (Ti2-A, Nikon).

### 7.11 Cell Viability Test of γδ T

The cell viability of γδ T cells in different cryogenic medium formulations was tested by staining dead T cells nucleus with propidium iodide (PI). Specifically, 5×10⁸ cells/mL of γδ T cells were cryopreserved with PBS with 2.5% (v/v) DMSO and 100 mM sucrose in a cryotube or APC-cryoMN. Conventional cryogenic medium of 10% DMSO and 90% FBS was used to compare the cell viability in a cryotube as the control group. After storage in liquid nitrogen for one month, cells were rapidly recovered in prewarmed FBS-free culture medium contained 1 μM PI and subsequently incubated at 37° C. under 5% CO₂ in the dark for 20 min. The cell suspension was washed by PBS buffer using a centrifugation speed of 1200 rpm for 10 min. The cell pellet was resuspended in 500 μL PBS buffer supplement with 1% BSA. The stained dead cells were then detected by a BD Biosciences benchtop Flow Cytometer and were analyzed by FlowJo software (TreeStar, version 10.5.3).

### 7.12 Surface Biomarker Change of γδ T after Cryopreservation

The surface biomarker change of γδ T cells was detected by the T cell receptor (TCR) Vδ2. After cryopreserved in PBS with 2.5% (v/v) DMSO and 100 mM sucrose for one month in liquid nitrogen, γδ T cells were recovered in prewarmed culture medium and resuspended with PBS buffer supplement with 1% BSA. TCR Vδ2 monoclonal antibody was applied on the γδ T cell suspension followed by the second antibody goat anti-mouse 488 after washing. The γδ T cells were then washed with PBS buffer and resuspended in 500 μL PBS buffer supplement with 1% BSA. The immunostained Vδ2 cells were then measured by a BD Biosciences benchtop Flow Cytometer and were analyzed by FlowJo software (TreeStar, version 10.5.3).

### 7.13 Mesothelioma Xenograft Mouse Model

NOD.Cg-Prkdc^{scid}Il2rg^{tm1Wjl}/SzJ NSG) mice were obtained from the City University of Hong Kong Laboratory Animal Research Unit. The construction of luciferase reporter mesothelioma cell lines referred to the reported work. The xenograft pleural mesothelioma-bearing mice were established by injecting 5×10⁶ MSTO-luc intrapleurally (i.pl) into NOD.Cg-Prkdc^{scid}Il2rg^{tm1Wjl}/SzJ (NSG) mice. Luciferase signals were monitored on the following day by NightOWL II LB 983 In Vivo Imaging System (Berthold Technologies). The mesothelioma xenograft mouse model was ready to use when the luciferase signals were detectable. 4 patches of Vδ2 T cell-loaded APC-cryoMNs (5×10⁶ cells per patch, a total of 2×10⁷ cells) were applied directly on the skin of each mouse for one dose treatment. After the application of each dose, the application site was covered with Tegaderm™ film (3M) for protection. On the same days, 1×10⁷ Vδ2 T cells in 100 μL PBS were injected into another group of mice intravenously (i.v.), and 100 μL of PBS was administered to the control group.

### 7.14 In Vivo Tracking of Vδ2 T Cell Delivered by APC-cryoMNs

For the tracking of Vδ2 T cells in vivo, mice were sacrificed at 1-4 days post one dose Vδ2 T cells injection. The skin tissue was collected and embedded in a cryomatrix embedding medium and frozen by liquid nitrogen for the following cryosection immunostaining analysis. The spleen, lung, tumor, and liver tissues were collected and processed by grinding them through a 70 μm cell strainer after treating them with red blood cell lysis buffer. The cell suspensions were then incubated for 30 min in the dark, followed by washing them twice with FACS buffer. After that, incubating with conjugated antibodies CD3 and Vδ2 TCR in 100 μL FACS buffer (1% FBS in PBS) at 4° C. for 30 min for surface protein staining. After washing the cells with FACS buffer, cells were then measured by a flow cytometer (BD FACSymphony A1).

### 7.15 Mesothelioma Treatment Using Vδ2 T Cell Loaded APC-cryoMNs

Both 1-dose and 2-dose of Vδ2 T cell-loaded APC-cryoMNs were applied on mesothelioma-bearing mice on day 3 as well as day 3 and 6 post tumor injection respectively. Vδ2 T cells were injected intravenously as i.v. group, while PBS was used as control group. Mesothelioma luciferase activities of APC-cryoMN (n=3 for 1- and 2-dose), i.v. (n=4 for 1-dose; n=3 for 2-dose), and control groups (n=4) were measured before and after treatment on day 2, 5, and 9 post tumor injection by NightOWL II LB 983 In Vivo Imaging System (Berthold Technologies). The mice weight was measured on day 0, 2, 5, and 9. The survival of mice was monitored on day 2, 5, and 9 post tumor injection until the experimental endpoint.

### 7.16 Melanoma Treatment Using Vδ2 T Cell Loaded APC-cryoMNs

For melanoma bearing mice, 1×107 A375 cells were injected into the right back flank of the mice subcutaneously (s.c.). Three doses of Vδ2 T cell-loaded APC-cryoMNs (5×10⁶ cells per patch, 4 patches of 2×10⁷ cells for one dose) were applied directly on the skin of each mouse on day 4, 6, and 8 post tumor injection respectively, followed by covering the application site with Tegaderm™ film (3M) for protection. The i.v. (n=5) and control (n=4) groups were administrated to compare with APC-cryoMN group (n=4). The weight of each mouse was monitored. The tumor length and tumor width of the mice were measured every 2-3 days until the experimental endpoint. Tumor volume (mm³) was calculated by the equation of

$$\frac{( length \times width \times width )}{2} .$$

Mice were sacrificed on day 11 post tumor injection. Dissected tumors and skin were embedded into a cryomatrix embedding medium and frozen by liquid nitrogen for the following histological and immunostaining analysis.

### 7.17 Histological Analysis

Frozen tumor and skin tissue was cryosectioned into 10 μm thick slices placed onto adhesive microscope glass slides by a cryostat (Cryostar NX70, ThermoFisher). The sectioned tissue was fixed and dehydrated, followed by staining with a H&E kit according to the provided protocol. The H&E stained tissue slides were imaged by a microscope (Ti2-A, Nikon).

For the immunostaining, the sliced tissue was fixed with acetone and placed in antigen retrieval buffer at 95° C. for 10 min. 10% normal goat serum was added to block for 30 min. The sliced tissue was stained with unconjugated primary TCR V delta 2 monoclonal antibody and subsequently conjugated secondary antibody. Mounting medium with DAPI was applied to stain the cell nucleus. The stained tissue sections were imaged with a confocal laser scanning microscope (SP8LIA++TRUE, LEICA).

### 7.18 Statistical Analysis

Statistical analyses were performed using One-way analysis of variance (ANOVA) to compare at least three groups and getting the P-value and Student's t test to obtain P-value significance between the two groups unless otherwise specified. P<0.05 is considered statistically significant (*P<0.05, **P<0.01, ***P<0.001, ****P<0.0001). Each experiment was repeated at least three times in triplicate unless otherwise indicated.

The exemplary embodiments are thus fully described. Although the description referred to particular embodiments, it will be clear to one skilled in the art that the invention may be practiced with variation of these specific details. Hence this invention should not be construed as limited to the embodiments set forth herein.

While the embodiments have been illustrated and described in detail in the foregoing description, the same is to be considered as illustrative and not restrictive in character, it being understood that only exemplary embodiments have been shown and described and do not limit the scope of the invention in any manner. It can be appreciated that any of the features described herein may be used with any embodiment. The illustrative embodiments are not exclusive of each other or of other embodiments not recited herein. Accordingly, the invention also provides embodiments that comprise combinations of one or more of the illustrative embodiments described above. Modifications and variations of the invention as herein set forth can be made without departing from the spirit and scope thereof, and, therefore, only such limitations should be imposed as are indicated by the appended claims.

1. A microneedle comprising a base and a tip distal from the base, wherein the microneedle is formed with an anisotropic porous composition comprising a plurality of channels extending in a substantially uniform direction through the microneedle from a base surface towards an outer surface defined by the tip, and wherein the plurality of channels are adapted to enable flow of a fluid therein.

2. The microneedle according to claim 1, wherein each of the plurality of channels has a substantially uniform channel diameter.

3. The microneedle according to claim 2, wherein the channel diameter is about 20 μm; or about 30 μm; or between 20-50 μm; or between 20-150 μm; or between 85-145 μm.

4. The microneedle according to claim 1, wherein the flow of the fluid within the plurality of channels is unidirectional.

5. The microneedle according to claim 4, wherein the plurality of channels have a first physical property when the fluid flows into the microneedle, and a second physical property when the fluid flows out of the microneedle, and wherein the first physical property is different from the second physical property.

6. The microneedle according to claim 5, wherein the first and second physical properties include morphological property, mechanical property, pore structure, and young's modulus.

7. The microneedle according to claim 1, wherein the anisotropic porous composition is formed with a cross-linked polymeric matrix.

8. The microneedle according to claim 7, wherein the cross-linked polymeric matrix includes a plurality of monomers selected from a group consisting of gelatin, alginate, polyvinyl alcohol (PVA), poly 2-hydroxyethylmethacrylate (PHEMA), polyacrylamide (PAAm), vinylgroup-modified hyaluronic acid (HA), methactylated hyaluronic acid (MeHA) and a combination thereof.

9. A microneedle patch comprising a substrate and at least a microneedle according to claim 1 disposed on the substrate.

10. The microneedle patch according to claim 9, wherein the substrate is unitary formed with the microneedle, and wherein the plurality of channels extend through both the substrate and the microneedle, from an outer surface of the substrate towards the outer surface defined by the tip of the microneedle.

11. The microneedle patch according to claim 9, wherein the substrate is separately formed with the microneedle, and is attached to the base of the microneedle.

12. The microneedle patch according to claim 11, wherein the substrate is a metal sheet.

13. The microneedle patch according to claim 11, wherein the substrate is a handle.

14. The microneedle patch according to claim 9, further comprising an electrochemical sensor attached to the substrate.

15. The microneedle patch according to claim 9, further comprising a payload pre-loaded within the microneedle patch.

16. The microneedle patch according to claim 15, wherein the payload is selected from a group consisting of a cell, a drug, an extracellular vesicle, a macromolecule, and a combination thereof.

17. The microneedle patch according to claim 16, wherein the cell is selected from a group consisting of an immune cell, an antigen cell, a stem cell, a fibroblast, a melanocyte, a hair follicle cell, a beta cell, a therapeutic cell, a prophylactic cell, and a combination thereof.

18. The microneedle patch according to claim 16, wherein the extracellular vesicle is selected from a group consisting of an exosome, a microvesicle, an apoptotic body, an autophagic extracellular vesicle, a matrix vesicle, a stressed extracellular vesicle, and a combination thereof.

19. The microneedle patch according to claim 16, wherein the macromolecule is selected from a group consisting a genetic material, a polypeptide, a protein, a deoxyribonucleic acid sequence (DNA), a ribonucleic acid sequence (RNA), an enzyme, an antibody, and a combination thereof.

20. The microneedle patch according to claim 9, comprising a plurality of microneedles forming a microneedle array disposed on the substrate.

21. A method of manufacturing a microneedle, comprising the steps of:
(i) casting a pre-polymer solution comprising a plurality of monomers into a mold defined with at least a recess shaped in a microneedle structure, wherein the microneedle structure comprising a base and a tip distal from the base;
(ii) freezing the pre-polymer solution with a temperature gradient across the mold;
(iii) cross-linking the plurality of monomers to form a cross-linked polymer matrix;
(iv) lyophilising the cross-linked polymer matrix to form an anisotropic porous composition defined with the microneedle structure, wherein the anisotropic porous composition comprising a plurality of channels extending in a substantially uniform direction through the anisotropic porous composition from a base surface towards the outer surface of defined by the tip; and
(v) removing the anisotropic porous composition from the mold.

22. The method according to claim 21, wherein the temperature gradient is provided by arranging a first surface of the mold at room temperature; or at a temperature between 4 to 28° C.; or at a temperature between 18 to 24° C., and further arranging a second surface of the mold opposite the first surface at a temperature between −0 to −300° C.; or at a temperature between −25 to −285° C.; or at a temperature between −50° C. to −250° C.

23. The method according to claim 22, wherein the second surface of the mold is placed in contact with a cooling element selected from a group consisting of liquid nitrogen, liquid helium, dry ice, and a mixture thereof.

24. The method according to 23, wherein the cooling element is in form of a cooling bath.

25. The method according to claim 21, wherein the cross-linking of the plurality of monomers is achieved by freezing the pre-polymer solution at a temperature below zero.

26. The method according to claim 21, wherein the cross-linking of the plurality of monomers is achieved by exposing the pre-polymer solution to a UV source.

27. The method according to claim 21, further comprising a step of thawing the cross-linked polymer matrix at room temperature before the lyophilizing step (iv).

28. The method according to claim 27, wherein the cross-linked polymer matrix is thawed in deionized water for an 1 hour to 3 days; or for 4 hours to 2 days; or for 6 hours to 36 hours.

29. The method according to claim 21, wherein the mold is formed with a mold material selected from a first group consisting of a polymer, a metal, and a combination thereof; or from a second group consisting of polydimethylsiloxane (PDMS), steel, resin, and a combination thereof.

30. The method according to claim 21, wherein each of the plurality of channels has a substantially uniform channel diameter.

31. The method according to claim 30, further comprising a step of adjusting the channel diameter by varying a polymer concentration of the pre-polymer solution.

32. The method according to claim 30, further comprising a step of adjusting the channel diameter by varying the temperature gradient.

33. The method according to claim 30, further comprising a step of adjusting the channel diameter to about 20 μm; or about 30 μm; or between 20-50 μm; or between 20-150 μm; or between 85-145 μm.

34. The method according to claim 21, wherein the plurality of monomers are selected from a group consisting of gelatin, alginate, polyvinyl alcohol (PVA), poly 2-hydroxyethylmethacrylate (PHEMA), polyacrylamide (PAAm), vinylgroup-modified hyaluronic acid (HA), methactylated hyaluronic acid (MeHA) and a combination thereof.

35. A method of manufacturing a microneedle patch, comprising the steps of the manufacturing method of a microneedle according to claim 21, and further comprising a step of forming a substrate on which at least a microneedle is disposed.

36. The method according to claim 35, wherein the substrate is formed by casting the pre-polymer solution into the mold beyond filling the recess of the mold along a mold wall.

37. The method according to claim 35, wherein the substrate is formed by attaching a separate layer to the base of the microneedle.

38. The method according to claim 37, wherein the separate layer is a metal sheet.

39. The method according to claim 37, wherein the separate layer is a handle.

40. The method according to claim 35, further comprising a step of loading a payload to the microneedle patch.

41. The method according to claim 40, wherein the payload is selected from a group consisting of a cell, a drug, an extracellular vesicle, a macromolecule, and a combination thereof.

42. The method according to claim 41, wherein the cell is selected from a group consisting of an immune cell, an antigen cell, a stem cell, a fibroblast, a melanocyte, a hair follicle cell, a beta cell, a therapeutic cell, a prophylactic cell, and a combination thereof.

43. The microneedle patch according to claim 41, wherein the extracellular vesicle is selected from a group consisting of an exosome, a microvesicle, an apoptotic body, an autophagic extracellular vesicle, a matrix vesicle, a stressed extracellular vesicle, and a combination thereof.

44. The method according to claim 41, wherein the macromolecule is selected from a group consisting a genetic material, a polypeptide, a protein, a deoxyribonucleic acid sequence (DNA), a ribonucleic acid sequence (RNA), an enzyme, an antibody, and a combination thereof.

45. The method according to claim 40, wherein the payload is loaded to the pre-polymer solution when the polymer solution is casted in the mold.

46. The method according to claim 40, wherein the payload is loaded to the anisotropic porous composition before removing from the mold.

47. The method according to claim 35, further comprising a step of attaching an electrochemical sensor to the substrate.

48. A method of delivery of a payload comprising the step of applying the microneedle patch of claim 15 to an area of application to deliver the payload from the microneedle patch to the area of application.

49. The method according to claim 48, wherein the area of application is a skin surface.

50. The method according to claim 48, wherein the area of application is an ocular surface.

51. The method according to claim 48, wherein the area of application is an oral surface.

52. A method of collecting a sample comprising the step of contacting the sample with the microneedle patch of claim 9 to collect the sample.

53. The method of claim 52, wherein the sample is a tear.
